(19) United States
(12) Patent Application Publication
(10) Pub. No.: US 2025/0001213 A1
(43) Pub. Date: Jan. 2, 2025

(54) TREATMENT SYSTEMS

(71) Applicant: SHANGHAI UNITED IMAGING HEALTHCARE CO., LTD., Shanghai (CN)

(72) Inventors: Cheng NI, Shanghai (CN); Peng WANG, Shanghai (CN); Shoubo HE, Shanghai (CN); Feichao FU, Shanghai (CN)

(73) Assignee: SHANGHAI UNITED IMAGING HEALTHCARE CO., LTD., Shanghai (CN)

(21) Appl. No.: 18/759,818

(22) Filed: Jun. 29, 2024

(30) Foreign Application Priority Data
Jun. 29, 2023 (CN) 202310788992.8

(51) Int. Cl. A61N 5/10

(52) U.S. Cl. CPC A61N 5/1084; A61N 5/1049; A61N 5/1082; A61N 2005/1055

(57) ABSTRACT

The present disclosure provides a treatment system including a radiotherapy device, a magnetic resonance imaging (MRI) device, and a support. The radiotherapy device is configured to emit a radiation beam. The MRI device may include a magnet assembly for providing a magnet field in an accommodation space. The support may be configured to be connected with the radiotherapy device, and the radiotherapy device may be configured to rotate around at least one rotation axis.

### CROSS-REFERENCE TO RELATED APPLICATIONS

This application claims priority to Chinese Patent Application No. 202310788992.8, filed on Jun. 29, 2023, the entire contents of which are hereby incorporated by reference.

### TECHNICAL FIELD

This specification relates to the field of medical devices, and in particular, to a treatment system.

### BACKGROUND

The Gamma Knife is more effective in brain tumors, with the advantages of high precision, safety, and reliability, non-invasiveness, etc. Currently, online imaging in a Gamma Knife system may be achieved by X-ray imaging or magnetic resonance imaging (MRI). For soft tissues such as the brain, X-ray imaging may have the disadvantage of lower contrast compared to MRI.

Therefore, it is desirable to provide a treatment system to combine MRI with the Gamma Knife system to enable precise online localization and online treatment of a subject (e.g., a lesion).

### SUMMARY

One or more embodiments of the present disclosure provide a treatment system including a radiotherapy device, a magnetic resonance imaging (MRI) device, and a support. The radiotherapy device may be configured to emit a radiation beam. The MRI device may include a magnet assembly for providing a magnet field in an accommodation space. The support may be configured to be connected with the radiotherapy device, and the radiotherapy device may be configured to rotate around at least one rotation axis.

In some embodiments, an angle between a direction of the magnet field in the accommodation space and a direction of the radiation beam may be equal to or less than 90 degrees.

In some embodiments, the at least one rotation axis may include at least one of a first axis parallel with an extension direction of a table of the MRI device in the accommodation space or a second axis perpendicular to the extension direction of the table of the MRI device in the accommodation space.

In some embodiments, a rotation angle of the radiotherapy device rotating around the first axis may be 360 degrees.

In some embodiments, the radiotherapy device may be configured to rotate around the first axis with a rotation of the support.

In some embodiments, the radiotherapy device may be configured to rotate around the first axis with a rotation of the MRI device.

In some embodiments, the radiotherapy device is configured to rotate around one of at least one rotation axis independently of the support.

In some embodiments, a rotation angle of the radiotherapy device rotating around the second axis may be less than 180 degrees.

In some embodiments, the second axis may be perpendicular or parallel to a surface of the table where a subject to be treated, and the radiotherapy device may be arranged on the support such that a direction of the radiation beam may be perpendicular or substantially perpendicular to a plane defined by the second axis and the first axis.

In some embodiments, the at least one rotation axis may include a third axis, an angle between the third axis and the second axis is less than 90 degrees.

In some embodiments, the support includes a rotating shell, wherein the rotating shell in a semicircular shape is configured to provide the support to the radiotherapy device.

In some embodiments, the magnet assembly may include a first magnet and a second magnet separately provided at two sides of the accommodation space along a direction parallel to a direction of the magnet field.

In some embodiments, a direction of the radiation beam may be parallel to or substantially parallel to the direction of the magnet field.

In some embodiments, a direction of the radiation beam may be perpendicular to or substantially perpendicular to the direction of the magnet field.

In some embodiments, the at least one rotation axis may include a first axis perpendicular to a direction of the magnet field in the accommodation space and a second axis perpendicular to or parallel with the direction of the magnet field in the accommodation space.

In some embodiments, a guiding slot may be provided on a gantry of the MRI device and configured to pass the radiation beam, and/or a recess is provided on each of the first magnet and the second magnet which are configured to pass the radiation beam.

In some embodiments, the magnet assembly may include one or more magnets each of which is in a tubular structure and surrounds the accommodation space.

In some embodiments, the at least one rotation axis may include a first axis parallel with a direction of the magnet field in the accommodation space and a second axis perpendicular to the direction of the magnet field in the accommodation space.

In some embodiments, the magnet assembly may include a first section and a second section, and a size of the first section may be smaller than a size of the second section.

In some embodiments, the magnet assembly may include one or more magnets each of which is in a tubular structure to form the accommodation space and a third magnet. The one or more magnets and the third magnet may be located on two sides of the radiotherapy device and the support along a direction of the magnet field.

### BRIEF DESCRIPTION OF THE DRAWINGS

The present disclosure is further illustrated in terms of exemplary embodiments. These exemplary embodiments are described in detail with reference to according to the drawings. These embodiments are non-limiting exemplary embodiments, in which like reference numerals represent similar structures, and wherein:

FIG. 1 is a diagram illustrating an exemplary treatment system according to some embodiments of the present disclosure;

FIG. 2A is a schematic diagram illustrating a radiotherapy system based on X-ray imaging according to some prior art;

FIG. 2B is a schematic diagram illustrating a radiotherapy system based on X-ray imaging according to yet other prior art;

FIG. 3 is a diagram illustrating an exemplary treatment apparatus according to some embodiments of the present disclosure;

FIG. 4A is a schematic diagram illustrating an exemplary treatment system according to some embodiments of the present disclosure;

FIG. 4B is a schematic diagram illustrating a section view of an exemplary treatment system according to some embodiments of the present disclosure;

FIG. 4C is a schematic diagram illustrating another exemplary treatment system according to some embodiments of the present disclosure;

FIG. 5A is a schematic diagram illustrating an exemplary treatment system according to some embodiments of the present disclosure;

FIG. 5B is a schematic diagram illustrating an treatment system according to some embodiments of the present disclosure;

FIG. 5C is a schematic diagram illustrating another exemplary treatment system according to some embodiments of the present disclosure;

FIG. 6A is a schematic diagram illustrating an exemplary treatment system according to some embodiments of the present disclosure;

FIG. 6B is a schematic diagram illustrating a section view of an exemplary treatment system according to some embodiments of the present disclosure;

FIG. 6C is a schematic diagram illustrating an exemplary treatment system according to some embodiments of the present disclosure;

FIG. 6D is a schematic diagram illustrating an exemplary treatment system according to some embodiments of the present disclosure; and

FIG. 6E is a schematic diagram illustrating a traverse section of an exemplary treatment system according to some embodiments of the present disclosure.

### DETAILED DESCRIPTION

To more clearly illustrate the technical solutions related to the embodiments of the present disclosure, a brief introduction of the drawings referred to the description of the embodiments is provided below. The accompanying drawings do not represent the entirety of the embodiments.

It should be understood that “system”, “device”, “unit” and/or “module” as used herein is a manner used to distinguish different components, elements, parts, sections, or assemblies at different levels. However, if other words serve the same purpose, the words may be replaced by other expressions.

As shown in the present disclosure and claims, the words “one”, “a”, “a kind” and/or “the” are not especially singular but may include the plural unless the context expressly suggests otherwise. In general, the terms “comprise,” “comprises,” “comprising,” “include,” “includes,” and/or “including,” merely prompt to include operations and elements that have been clearly identified, and these operations and elements do not constitute an exclusive listing. The methods or devices may also include other operations or elements.

When describing the operations performed in the embodiments of the present disclosure in terms of steps, the order of the steps is all interchangeable if not otherwise indicated, the steps are omissible, and other steps may be included in the operation.

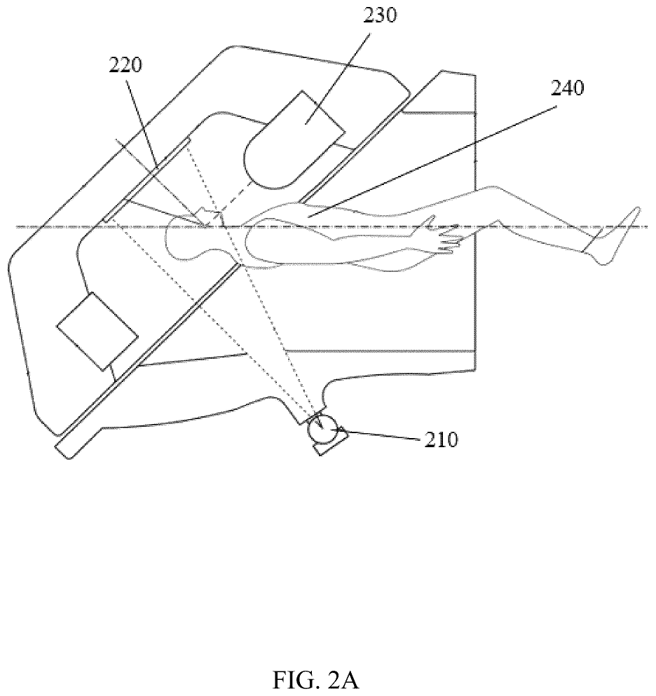

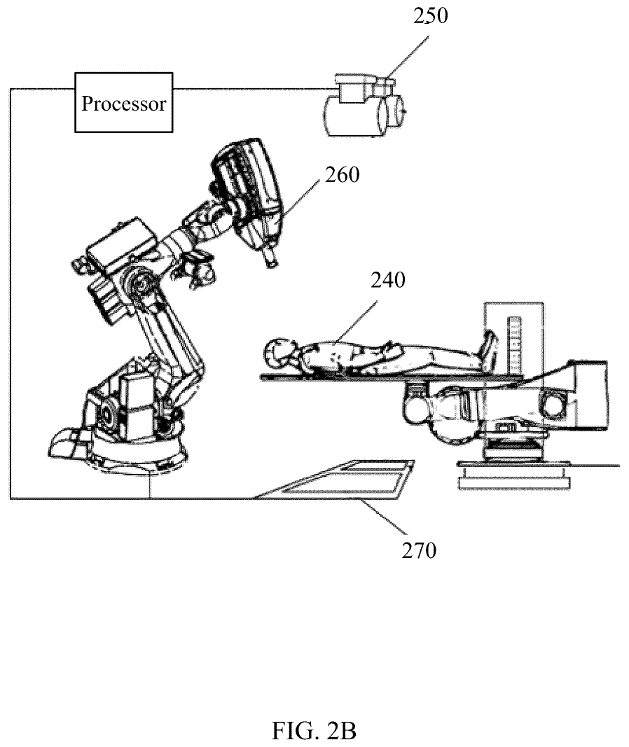

A gamma knife treatment system based on X-ray imaging may include an X-ray imaging assembly and a treatment assembly. The X-ray imaging assembly may include an X-ray source and a detector. For example, FIG. 2A illustrates an exemplary gamma ray radiotherapy system. As shown in FIG. 2A, after X-rays emitted by a tube 210 pass through a subject 240, at least a portion of the X-rays may be detected by the detector 220, obtaining a medical image that carries information about the subject 240, and based on the medical image, the processor may control the gamma knife 230 to emit gamma rays to treat the subject 240. As another example, FIG. 2B illustrates another exemplary radiotherapy system. As shown in FIG. 2B, X-rays emitted by an X-ray source 250 and passing through the subject 240 may be received by the detector 270 to obtain a medical image that carries information about the subject 240. Based on the medical image, the processor may control a gamma knife 260 to emit gamma rays to treat the subject 240. Compared to magnetic resonance imaging (MRI), for soft tissues such as the brain, X-ray imaging has the disadvantage of low contrast. The combination of a gamma knife system with MRI is usually large in size, and it is difficult to fixedly integrate an MRI device with a gamma knife device to achieve synchronous rotation, thus the treatment may not be performed in a 360° around the brain.

Embodiments of the present disclosure provide a treatment system combining an imaging device and a radiotherapy device in gamma knife as a single unit and including the radiotherapy device capable of rotating around at least one rotation axis. For example, the radiotherapy device may rotate around an axis of the imaging device. The axis may be parallel to the extension direction of a table in a treatment space of the treatment system or perpendicular to the transverse section of a subject located on the table in the treatment space of the treatment system. As another example, the radiotherapy device may rotate around an axis perpendicular to the axis of the imaging device. The radiotherapy device may rotate with a rotation of the imaging device (e.g., the MRI device) or rotate relative to the imaging device (e.g., the MRI device) such that the imaging device (e.g., the MRI device) and the radiotherapy device may be interlinked and controlled. In some embodiments, the imaging device may include the MRI device. The magnetic resonance imaging device may include a magnet assembly for providing a magnet field. The radiotherapy device may around an axis perpendicular to a direction of the magnet field and/or an axis parallel to the direction of the magnet field. For example, when the radiotherapy device is located at the top portion of the magnetic resonance imaging device, and the direction of the magnet field is parallel to the extension direction of the table of the treatment system in the treatment space, the radiotherapy device may rotate around a first horizontal axis (i.e., a first axis) parallel to the direction of the magnet field and a second horizontal axis (i.e., a second axis) perpendicular to the direction of the magnet field (or the first horizontal axis). When the direction of the magnet field is perpendicular to the extension direction of the table of the treatment system in the treatment space, the radiotherapy device may rotate around a first horizontal axis (i.e., a first axis) perpendicular to the direction of the magnet field and a second horizontal axis (i.e., a second axis) perpendicular to the direction of the magnet field (or the first horizontal axis). As another example, when the radiotherapy device is located at the side portion of the magnetic resonance imaging device and the direction of the magnet field is parallel to the extension direction of the table of the treatment system in the treatment space, the radiotherapy device may rotate around a first horizontal axis (i.e., the first axis) parallel to the direction of the magnet field and a vertical axis (i.e., a second axis) perpendicular to the direction of the magnet field. When the direction of the magnet field is perpendicular to the extension direction of the table of the treatment system in the treatment space, the radiotherapy device may rotate around a first horizontal axis (i.e., the first axis) perpendicular to the direction of the magnet field and a vertical axis parallel (i.e., the second axis) to the direction of the magnet field. The MRI device may have the advantage of high imaging contrast, which allows for online localization of a subject (e.g., a lesion), which in turn allows the radiotherapy device to perform an online treatment on the subject based on a magnetic resonance image.

In addition, the radiotherapy device of the treatment system may rotate around an axis perpendicular to a traverse section of the subject from 0° to 360° (i.e., a treatment angle of the radiotherapy device may be 360° in the traverse section of the subject), resulting in a wider treatment range and a higher treatment efficiency. In some embodiments, the radiotherapy device may rotate around at least two axes (i.e., the first axis and the second axis). For example, the radiotherapy device may rotate around a rotation axis of the MRI device and an axis perpendicular to the rotation axis of the MRI device such that the treatment angle of the subject may be a stereo angle (i.e., the treatment angle may be less than 90° in a sagittal plane or a coronal plane of the subject), thereby allowing the treatment on the subject more precise. In some embodiments, an angle between a direction of a radiation beam of the radiotherapy device and a direction of the magnet field may change with the rotation of the radiotherapy device around the at least two axes. Moreover, different ray doses may be taken at different angles between the direction of the radiation beam and the direction of the magnet field, reducing damage to organs other than subject (e.g., mouth, nose, etc.). Descriptions of the traverse section, the sagittal plane, and the coronal plane may be found hereinafter and may not be repeated here.

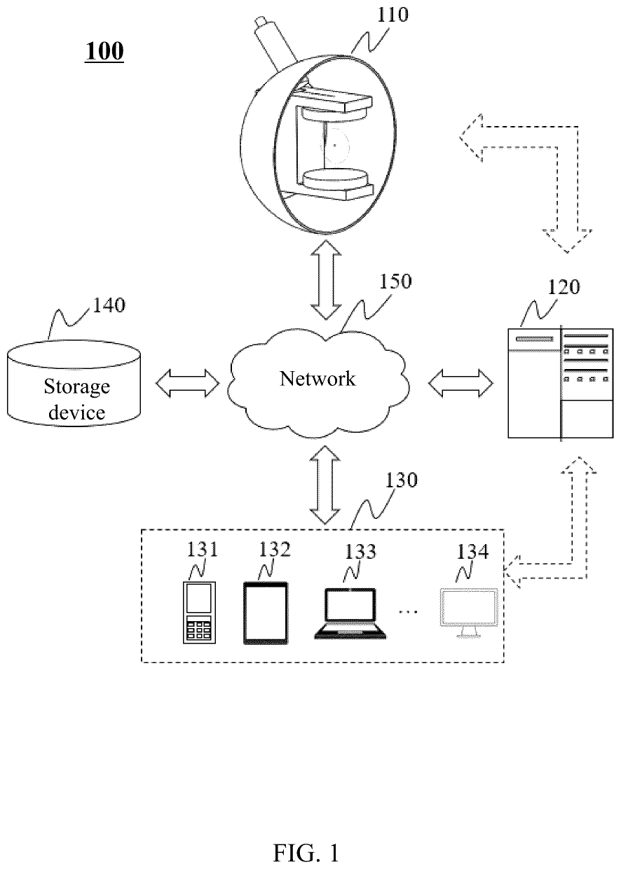

FIG. 1 is a diagram illustrating an exemplary treatment system according to some embodiments of the present disclosure.

As shown in FIG. 1, a treatment system 100 may include a treatment apparatus 110 a processing device 120, one or more terminals 130, a storage device 140, and a network 150. Assemblies in the treatment system 100 may be connected in one or more of a plurality of ways. Merely by way of example, the treatment apparatus 110 may be connected to the processing device 120 via the network 150, as shown in FIG. 1. As another example, the treatment apparatus 110 may be directly connected to the processing device 120, e.g., the treatment apparatus 110 and the processing device 120 may be connected as indicated by the dashed bi-directional arrows in FIG. 1. As yet another example, the storage device 140 may be connected directly to the processing device 120 (not shown in FIG. 1) or via the network 150. As yet another example, one or more terminals 130 may be connected directly to the processing device 120 (as indicated by the dashed bidirectional arrows connecting the one or more terminals 130 to the processing device 120) or via the network 150.

In some embodiments, the treatment system 110 may include a radiotherapy device, an imaging device, and a support.

In some embodiments, the imaging device may be configured to image a subject to obtain one or more images of the subject. The one or more images of the subject may be used for a guidance of treatment of the subject, for example, positioning of the subject. In some embodiments, the subject may include a biological subject and/or a non-biological subject. In some embodiments, the subject may include a patient or an animal. In some embodiments, the subject may include a specific part of a human body, such as the head. The imaging device may include a computed tomography (CT) device, a positron emission tomography (PET) device, a magnetic resonance imaging (MR) device, an X-ray imaging device, or the like.

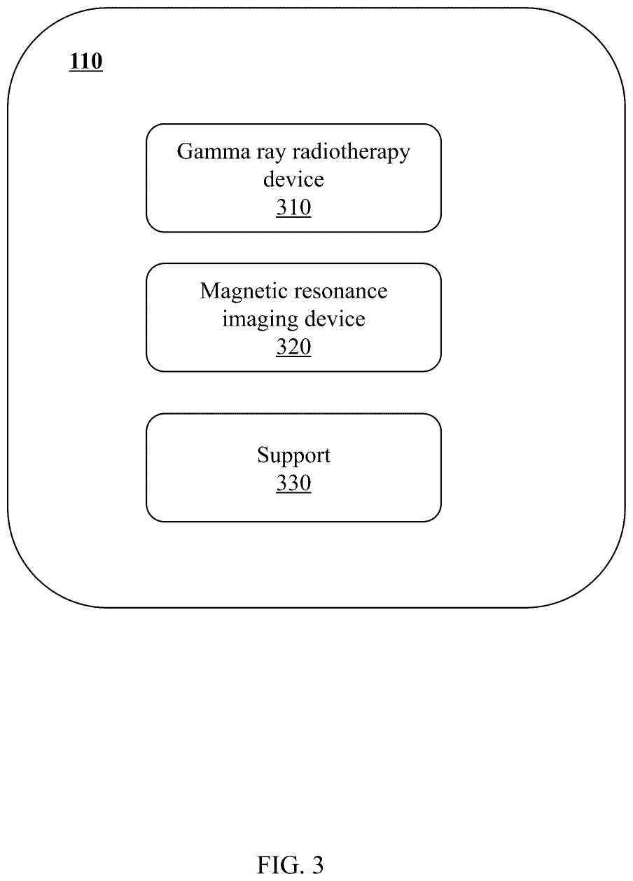

In some embodiments, the radiotherapy device has a field value of less than 1.5 T. In some embodiments, the radiotherapy device may rotate around at least one rotation axis. For example, the radiotherapy device may rotate around at least two rotation axes. In some embodiments, the at least one axis may include an X-axis perpendicular to a traverse section of the subject when the subject is located in the treatment space of the treatment system 100. A rotation angle of the radiotherapy device around the X-axis may be within a range of 0 degree-360 degrees, or a range of 0 degrees-270 degrees, or a range of 0 degrees-180 degrees. The direction of the X-axis may be parallel to an extension direction of a table of the treatment system in the treatment space (i.e., a long axis direction of the table in the treatment space). In some embodiments, the at least one rotation axis may include the X-axis perpendicular to the traverse section of the subject and a Z-axis perpendicular to a sagittal plane of the subject when the subject is located in the treatment space of the treatment system 100. In some embodiments, the direction of the Z-axis may be parallel to a short axis direction of the table of the treatment system in the treatment space. In some embodiments, a rotation angle of the radiotherapy device around the Z-axis may be within a range of 0 degrees-90 degrees or a range of 0 degrees-45 degrees, etc. In some embodiments, the radiotherapy device may be configured to perform online treatment on the subject based on localization guidance from the MRI device. In some embodiments, the radiotherapy device may be a gamma knife. In some embodiments, the support may be configured to provide support for the magnetic resonance imaging device and/or the radiotherapy device. In some embodiments, the support may include a gantry of the imaging device. The support may be configured to rotate around the X axis of the treatment apparatus 110. The rotation of the support may cause the rotation of the radiotherapy device and the imaging device. More descriptions regarding the treatment system may be found in FIG. 3 and related description thereof.

In some embodiments, data (e.g., a magnetic resonance image, etc.) obtained by the treatment system 110 may be transmitted to the processing device 120 for further analysis. Additionally or alternatively, the data obtained by the treatment system 110 may be sent to a terminal device (e.g., the one or more terminals 130) for display and/or a storage device (e.g., the storage device 140) for storage.

The processing device 120 may process data and/or information obtained and/or extracted from the treatment system 110, the one or more terminals 130, the storage device 140, and/or other storage devices. For example, the processing device 120 may obtain images from the treatment system 110. In some embodiments, the processing device 120 may control the treatment system 110. For example, the processing device 120 may control the radiotherapy device to rotate around an axis. As another example, the processing device 120 may control the radiotherapy device to move in a plane perpendicular to a direction of the magnet field of a magnet. As yet another example, the processing device 120 may control the radiotherapy device to rotate around an axis perpendicular to a traverse section of the subject.

In some embodiments, the processing device 120 may be a single server or a server group. The server group may be centralized or distributed. In some embodiments, the processing device 120 may be local or remote. In some embodiments, the processing device 120 may be implemented on a cloud platform. Merely by way of example, the cloud platform may include a private cloud, a public cloud, a hybrid cloud, a community cloud, a distributed cloud, an on-premises cloud, a multi-tier cloud, etc., or any combination thereof.

In some embodiments, the processing device 120 may be implemented on a computing device. In some embodiments, the processing device 120 may be implemented on a terminal (e.g., the one or more terminals 130). In some embodiments, the processing device 120 may be implemented on the treatment system 110. For example, the processing device 120 may be integrated in the one or more terminals 130 and/or the treatment system 110.

The one or more terminals 130 may be connected to the treatment system 110 and/or the processing device 120 for input/output of information and/or data. For example, a user may interact with the treatment system 110 via the one or more terminals 130 to control one or more components of the treatment system 110 (e.g., enter information about the subject, etc.). As another example, the treatment system 110 may output generated images to the one or more terminals 130 to display to a user.

In some embodiments, the one or more terminals 130 may include a mobile device 131, a tablet computer 132, a laptop computer 133, a desktop computer 134, etc., or any combination thereof. In some embodiments, the mobile device 131 may include a smart home device, a wearable device, a smart mobile device, a virtual reality device, an augmented reality device, etc., or any combination thereof. In some embodiments, one or more of the terminals 130 may remotely operate the treatment system 110. In some embodiments, the one or more terminals 130 may operate the treatment system 110 via a wireless connection. In some embodiments, the one or more terminals 130 may be part of the processing device 120. In some embodiments, the terminals 130 may be omitted.

The storage device 140 may store data and/or instructions. In some embodiments, the storage device 140 may store data obtained from the one or more terminals 130 and/or the processing device 120. For example, the storage device 140 may store magnetic resonance images, magnetic resonance imaging device parameters, treatment device parameters, etc. In some embodiments, the storage device 140 may store data and/or instructions that may be executed or used by the processing device 120 to perform the exemplary methods described in the present disclosure.

In some embodiments, the storage device 140 may include a mass storage device, a removable storage device, random access memory, read-only memory (ROM), etc., or any combination thereof.

The network 150 may include any suitable network capable of facilitating the exchange of information and/or data of one or more components in the application scenario 100. In some embodiments, one or more components in the application scenario 100 (e.g., the treatment system 110, one or more terminals 130, the processing device 120, or the storage device 140) may be in communication with one or more other components in the application scenario 100 to transmit information and/or data. In some embodiments, the network 150 may be any type of wired or wireless network or a combination thereof.

It should be noted that the above description of the treatment system is for illustrative purposes only and is not intended to limit the scope of the present disclosure. Various variations and modifications may be made in accordance with the present disclosure for those of ordinary skill in the art. However, these variations and modifications do not depart from the scope of the present disclosure. For example, the treatment system 110, the processing device 120, and the one or more terminals 130 may share a single storage device 140, or they may have separate storage devices.

FIG. 3 is a diagram illustrating an exemplary treatment apparatus according to some embodiments of the present disclosure.

In some embodiments, the treatment system 110 may include a radiotherapy device 310, an imaging device 320, and a support 330, as shown in FIG. 3.

The radiotherapy device 310 may be configured to emit a radiation beam. For example, the radiotherapy device 310 may emit a radiation beam with a higher radiation dose to a target region in a short period to provide high-dose irradiation to the target region. The imaging device 320 may include an X-ray imaging device, a digital radiography (DR) device, a computed radiography (CR) device, a digital fluorography (DF) device, a computed tomography (CT) device, a magnetic resonance imaging (MRI) device, a positron emission tomography (PET) imaging device, an emission computed tomography (ECT) imaging device, a magnetic resonance imaging (MR), positron emission tomography (PET), an ultrasound imaging device, etc., or any combination thereof.

In some embodiments, the magnetic resonance imaging device may include a magnet assembly, and the magnet assembly may be configured to provide a magnet field in an accommodation space or the treatment space of the treatment apparatus 300.

In some embodiments, the support 330 may be configured to provide a support for the imaging device 320 and/or the radiotherapy device 310. In some embodiments, the support 330 may include a rotation transmission component. In some embodiments, a rotating mechanism may be provided in the support 330, which may make the radiotherapy device 310 rotate around at least one rotation axis. The rotating mechanism may be of a plurality of structural forms. For example, the rotating mechanism may be a rotating shell, etc.

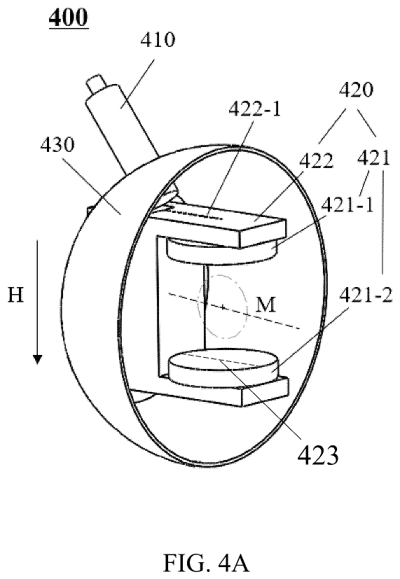

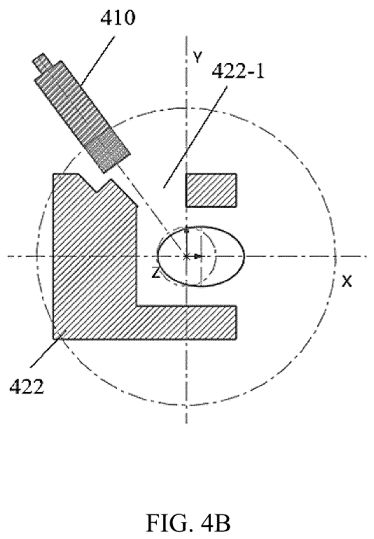

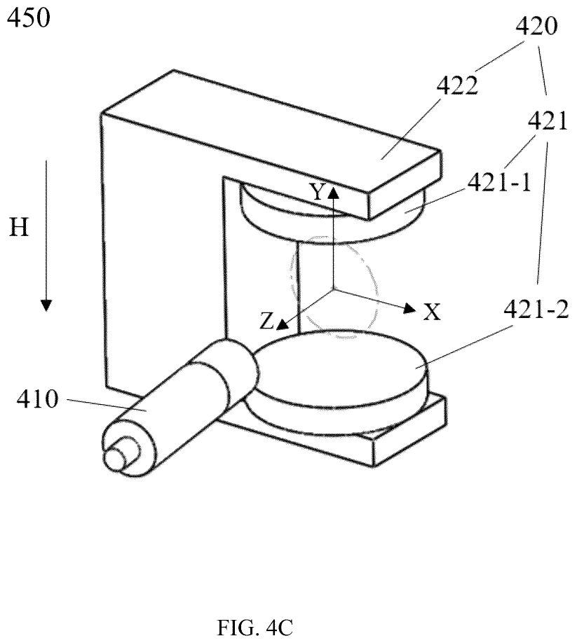

In some embodiments, a relative position of the radiotherapy device 310 to the imaging device 320 may include a plurality of forms. For example, the radiotherapy device 310 may be provided at an upper portion of the imaging device 320. As shown in FIG. 4A, the radiotherapy device 410 may be provided at an upper portion of the magnetic resonance imaging device 420. As another example, the radiotherapy device 310 may be provided on a side portion of the imaging device 320. As shown in FIG. 4B, the radiotherapy device 410 may be provided on the side portion of the magnetic resonance imaging device 420. In some embodiments, the radiotherapy device 310 may be rotated from the upper portion to the side portion of the imaging device 320.

In some embodiments, the radiotherapy device 310 may perform a radiation treatment on a subject based on one or more images obtained by the imaging device 320. In some embodiments, the radiotherapy device 310 may be of a plurality of forms such as a gamma knife, a linear gas pedal, etc.

In some embodiments, the radiotherapy device 310 may emit the radiation beam to the accommodation space to perform the radiation treatment on the subject. The radiation beam refers to a high-energy electromagnetic wave having a specific energy. The accommodation space refers to a spatial region configured to accommodate the subject, which is also known as a treatment space or an imaging space.

In some embodiments, when the imaging device 320 includes a magnetic resonance imaging (MRI) device, the radiotherapy device 310 may be provided on the support 330 such that an angle (also referred to as a default angle) between a direction of the magnet field of the magnet field in the accommodation space and a direction of the radiation beam emitted by the radiotherapy device 310 may be equal to or less than 90 degrees. As used herein, the default angle refers to an initial angle between the direction of the magnet field in the accommodation space and the direction of the radiation beam when the treatment apparatus 300 is in a non-operating state, for example, before the treatment apparatus 300 starts to work, or after the treatment apparatus 300 completes a task (e.g., an imaging scan or a treatment). The direction of the radiation beam emitted by the radiotherapy device 310 refers to a direction of a center axis of the radiation beam emitted by the radiotherapy device 310. In some embodiments, the angle between the direction of the magnet field of the magnet field in the accommodation space and the direction of the radiation beam emitted by the radiotherapy device 310 may change along the rotation of the radiotherapy device 310 and/or the imaging device 320. When the radiotherapy device 310 is provided on the support 330, the angle between the direction of the magnet field and the direction of the radiation beam emitted by the radiotherapy device 310 may be equal to or less than 90 degrees during the radiotherapy device 310 rotates 360 degrees around the X-axis. The radiotherapy device 310 rotates synchronously with the magnetic to keep the angle between the direction of magnetic field and the direction of the radiation beam less than or equal to 90 degrees. Also, the magnet and the radiotherapy device 310 can be mounted on the same support 330 to keep the angle less than 90 and rotated together.

In some embodiments, the radiotherapy device 310 may be arranged on the support 330 such that the angle between the direction of the magnet field in the accommodation space and the direction of the radiation beam emitted by the radiotherapy device 310 may be 40°. In some embodiments, the radiotherapy device 310 may be provided on the support 330 such that the angle between the direction of the magnet field in the accommodation space and the direction of the radiation beam emitted by the radiotherapy device 310 may be 50°. In some embodiments, the radiotherapy device 310 may be provided on the support 330 such that the angle between the direction of the magnet field in the accommodation space and the direction of the radiation beam emitted by the radiotherapy device 310 may be 60°. In some embodiments, the radiotherapy device 310 may be provided on the support 330 such that the angle between the direction of the magnet field in the accommodation space and the direction of the radiation beam emitted by the radiotherapy device 310 may be 70°. In some embodiments, the radiotherapy device 310 may be provided on the support 330 such that the angle between the direction of the magnet field in the accommodation space and the direction of the radiation beam emitted by the radiotherapy device 310 may be 90°.

In some embodiments, depending on the needs of the actual treatment, the radiotherapy device may emit the radiation beam with different doses when the direction of the radiation beam of the radiotherapy device is at a different angle to the direction of the magnet field. For example, when the angle between the direction of the radiation beam of the radiotherapy device and the direction of the magnet field is A1, the dose of the radiation beam emitted by the radiotherapy device 310 may be B1. When the angle between the direction of the radiation beam of the radiotherapy device and the direction of the magnet field is A2, the dose of the radiation beam emitted by the radiotherapy device 310 d may be B2. In some embodiments, a correspondence between the angle between the direction of the magnet field and the direction of the radiation beam and the dose of the radiation beam emitted by the radiotherapy device 310 may be preset. A dose corresponding to a specific angle between the direction of the magnet field and the direction of the radiation beam emitted by the radiotherapy device 310 may be determined based on the correspondence between the angle between the direction of the magnet field and the direction of the radiation beam and the dose of the radiation beam emitted by the radiotherapy device 310.

In some embodiments of the present disclosure, the angle between the direction of the radiation beam and the direction of the magnet field may be equal to or less than 90 degrees when the radiotherapy device 310 is provided on the support 330, and different doses of the radiation beam may be used when the angle between the direction of the radiation beam and the direction of the magnet field is different. Thus, damage to organs in other parts of the body other than subject (e.g., the mouth, the nose, etc.) may be reduced.

The radiotherapy device 310 may rotate around at least one axis (also referred to as a rotation axis). The at least one rotation axis may serve as a reference for the rotation of the radiotherapy device.

In some embodiments, the at least one rotation axis may include at least one of a first axis and a second axis. The first axis may be parallel with an extension direction of a table of the MRI device 320 in the accommodation space. The second axis may be perpendicular to the extension direction of the table of the MRI device 320 in the accommodation space.

As described in the present disclosure, the extension direction of the table of the MRI device 320 is taken as the X-axis (which may be perpendicular to the traverse section of the subject when the subject is lying down along the extension direction of the table). A direction perpendicular to the extension direction of the table of the MRI 320 and perpendicular to a surface of the table is taken as a Y-axis (which may be perpendicular to a sagittal plane of the subject when the subject is lying down along the extension direction of the table). A direction perpendicular to the extension direction of the table of the MRI 320 and parallel to the surface of the table as a Z-axis (which may be perpendicular to a coronal plane of the subject when the subject is lying down along the extension direction of the table). Thus, a reference coordinate system including the X axis, Y axis, and Z axis is constructed. In some embodiments, the first axis may be the X-axis and the second axis may be the Y-axis and/or the Z-axis. In some embodiments, the first axis may be a straight line parallel to the X-axis, and the second axis may be a straight line parallel to the Y-axis and/or a straight line parallel to the Z-axis.

The reference coordinate system may be set based on actual needs, which is not limited here. More descriptions regarding the support may be found in related descriptions hereinafter.

In some embodiments of the present disclosure, the radiotherapy device may enable precise treatment of the subject at a plurality of angles by rotating around at least one of the first axis or the second axis.

In some embodiments, when the imaging device is a magnetic resonance imaging (MRI) device, the rotation axis may be perpendicular and/or parallel to the direction of the magnet field of the magnet assembly. The direction of the magnet field may be parallel to the X-axis or the Y-axis.

In some embodiments, the second axis (i.e., parallel to the Y axis) may be perpendicular to the surface of the table where the subject is located, and the radiotherapy device 310 may be disposed on the support 330 such that the direction of the radiation beam may be perpendicular to, or approximately perpendicular to, a plane defined by the second axis and the first axis. In the embodiment, the second axis may be perpendicular to the surface of the table where the subject is located and may be perpendicular to the extension direction of the table of the magnetic resonance imaging device in the accommodation space. At this time, the plane defined by the second axis and the first axis may be perpendicular to the surface of the table. For example, the plane defined by the second axis and the first axis may be parallel to the sagittal plane of the subject (at this time, the subject is in a supine position on the surface of the table of the imaging device 320).

Merely by way of example, in the reference coordinate system constructed with the extension direction of the table of the imaging device 320 as the X-axis, the direction perpendicular to the extension direction of the table of the imaging device 320 and perpendicular to the surface of the table as the Y-axis, the direction perpendicular to the extension direction of the table of the imaging device 320 and parallel to the surface of the table as the Z-axis, the first axis may be the X-axis, and the second axis may be the Y-axis. At this point, the plane defined by the second axis and the first axis may be a plane defined by the X-axis and the Y-axis. The radiotherapy device 310 may be located at a plane parallel to or approximately parallel to the plane defined by the X-axis and the Z-axis. For example, an angle between the plane parallel where the radiotherapy device 310 is located and the plane defined by the X-axis and the Z-axis may be less than 45 degrees, or less than 30 degrees, or less than 20 degrees, or less than 10 degrees, etc. As used herein, the plane where the radiotherapy device 310 is located refers to a plane parallel to an extension direction or a long axis direction of the radiotherapy device 310.

In some embodiments of the present disclosure, by setting the second axis perpendicular to the surface of the table where the subject is located, and by setting the radiotherapy device on the support, it is possible to make the direction of the radiation beam perpendicular, or approximately perpendicular, to the plane defined by the second axis and the first axis.

In some embodiments, the second axis may be parallel to the surface of the table where the subject is located, and the radiotherapy device 310 may be provided on the support 330 such that the direction of the radiation beam may be perpendicular, or approximately perpendicular to, the plane defined by the second axis and the first axis. In the embodiment, the second axis may be parallel to the surface of the table where the subject is located and may be perpendicular to the extension direction of the table of the imaging device 320 in the accommodation space. The plane defined by the second axis and the first axis may be parallel to the surface of the table. For example, the plane defined by the second axis and the first axis may be parallel to the coronal plane of the d (at this point, the subject may be in the supine position on the surface of the table of the imaging device 320).

Merely by way of example, in the reference coordinate system constructed with the extension direction of the table of the imaging device 320 as the X-axis, the direction perpendicular to the extension direction of the table of the imaging device 320 and perpendicular to the surface of the table as the Y-axis, the direction perpendicular to the extension direction of the table of the imaging device 320 and parallel to the surface of the table as the Z-axis, the first axis may be the X-axis and the second axis may be the Z-axis, and the plane defined by the second axis and the first axis may be a plane defined by the X-axis and the Z-axis. The radiotherapy device 310 may be located at a plane parallel to or approximately parallel to the plane defined by the X-axis and the Y-axis. For example, an angle between the plane parallel where the radiotherapy device 310 is located and the plane defined by the X-axis and the Y-axis may be less than 45 degrees, or less than 30 degrees, or less than 20 degrees, or less than 10 degrees, etc.

In some embodiments of the present disclosure, by setting the second axis parallel to the surface of the table where the subject is located, and by setting the radiotherapy device on the support, the direction of the radiation beam may be made perpendicular or approximately perpendicular to the plane determined by the second axis and the first axis.

In some embodiments, the radiotherapy device may rotate around the at least two rotation axes. For example, the radiotherapy device may rotate around the first axis and the second axis. As a further example, the first axis may be the X axis and the second axis may be the Y axis. As still another example, the first axis may be the X axis and the second axis may be the Z axis.

In some embodiments, the at least one rotation axis may further include a third axis.

In some embodiments, the third axis may be perpendicular to the plane defined by the first axis and the second axis. For example, the first axis may be the X axis, the second axis may be the Y axis, the third axis may be the Z axis. As another example, the first axis may be the X axis, the second axis may be the Z axis, and the third axis may be the Y axis.

In some embodiments, the third axis may parallel to the plane defined by the first axis and the second axis and an angle between the third axis and the second axis may be less than 90 degrees. Merely by way of example, in the reference coordinate system constructed with the extension direction of the table of the imaging device 320 as the X-axis, the direction perpendicular to the extension direction of the table of the imaging device 320 and perpendicular to the surface of the table as the Y-axis, and the direction perpendicular to the extension direction of the table of the imaging device 320 and parallel to the surface of the table as the Z-axis, any straight line parallel to the plane defined by the first axis and the second axis and having an angle of less than 90 degrees with the Y-axis or the Z-axis may be determined as the third axis.

In some embodiments of the present disclosure, the radiotherapy device may also rotate around the third axis, thereby enabling treatment on any section of the subject.

In some embodiments, a rotation angle of the radiotherapy device 310 around the first axis may be 360 degrees. For example, in the reference coordinate system constructed with the extension direction of the table of the imaging device 320 as the X-axis, the direction perpendicular to the extension direction of the table of the imaging device 320 and perpendicular to the surface of the table as the Y-axis, and the direction perpendicular to the extension direction of the table of the imaging device 320 and parallel to the surface of the table as the Z-axis, the first axis may be the X-axis, the radiotherapy device 310 may rotate 360 degrees around the X-axis (i.e., the extension direction of the table) to achieve a 360° treatment in the traverse section of the subject. The traverse section may be perpendicular to the surface of the table where the subject is located. The traverse section may be defined by the X axis and the Y axis and is perpendicular to the surface of the table where the subject d is located.

In some embodiments, as the radiotherapy device 310 rotates around the first axis, the imaging device 320 may rotate synchronously with the radiotherapy device 310 to reduce the attenuation of treatment radiation rays.

In some embodiments of the present disclosure, the rotation angle of the radiotherapy device 310 rotating around the first axis may be 360 degrees, allowing for a wider treatment range and a higher treatment efficiency of the radiotherapy device 310.

In some embodiments, the radiotherapy device 310 may rotate around the first axis in a plurality of ways. For example, the radiotherapy device 310 may rotate around the first axis by way of a metric rotation. When the radiotherapy device 310 rotates around the first axis by way of the metric rotation, the first axis may pass through a rotation center of the radiotherapy device 310.

In some embodiments, the radiotherapy device 310 may be configured to rotate around the first axis with a rotation of the support 330. For example, the support 330 may rotate around the first axis, and the radiotherapy device 310 may be provided on the support 330 and connected to the support 330 such that the radiotherapy device 310 may rotate around the first axis with the rotation of the support 330. In some embodiment, the radiotherapy device 310 may rotate relative to the imaging device 320. In other words, the radiotherapy device 310 and the imaging device 320 may rotate separately.

In some embodiments of the present disclosure, the radiotherapy device may rotate around the first axis with the rotation of the support, allowing for a 360° treatment of the cross-section of the subject, resulting in a better treatment effect.

In some embodiments, the radiotherapy device 310 may be configured to rotate around one of at least one rotation axis independently of the support.

In some embodiments, the radiotherapy device 310 may be configured to rotate around the first axis with the rotation of the imaging device 320. For example, the magnetic resonance imaging device 320 may rotate synchronously with the rotation of the radiotherapy device 310.

In some embodiments of the present disclosure, the radiotherapy device 310 may rotate around the first axis with a rotation of the imaging device 320, i.e., the radiotherapy device 310 may rotate synchronously with the rotation of the imaging device 320 to reduce the attenuation of the radiation beam.

It should be noted that since the radiotherapy device 310 rotates around the first axis with the rotation of the imaging device 320, the imaging device 320 may rotate synchronously with the radiotherapy device 310, and the angle between the direction of the radiation beam of and the direction of the magnet field of the radiotherapy device 310 may be considered to remain constant.

In some embodiments, the radiotherapy device 310 may rotate around the second axis at a plurality of angles.

In some embodiments, the rotation angle of the radiotherapy device 310 rotating around the second axis may be less than 180 degrees, or less than 90 degrees, or less than 45 degrees, or less than 30 degrees. For example, in a reference coordinate system constructed in the reference coordinate system constructed with the extension direction of the table of the imaging device 320 as the X-axis, the direction perpendicular to the extension direction of the table of the imaging device 320 and perpendicular to the surface of the table as the Y-axis, and the direction perpendicular to the extension direction of the table of the imaging device 320 and parallel to the surface of the table as the Z-axis, the rotation angle of the radiotherapy device 310 rotating around the Y-axis or the Z-axis may be less than 180 degrees, or less than 90 degrees, or less than 45 degrees, or less than 30 degrees. As a further example, when the radiotherapy device 310 rotates around the Y-axis, a treatment angle may be in a plane perpendicular to the Y-axis (i.e., a plane defined by the X-axis and Z-axis), and the treatment angle may be less than 90°. When the radiotherapy device 310 rotates around the Z-axis, the treatment angle in the sagittal plane of the subject (i.e., the plane in which the X-axis and Y-axis are located) may be less than 90°.

In some embodiments of the present disclosure, the rotation angle of the radiotherapy device rotating around the second axis may be less than 180 degrees, allowing treatment of the sagittal plane or the coronal plane of the subject to be performed at an angle of less than 180 degrees, reducing the attenuation of the treatment radiation rays. At the same time damage to other parts of the organs of the subject (e.g., the mouth, the nose, etc.) may be reduced, thereby allowing a more precise treatment on the subject.

In some embodiments, the rotation angle of the radiotherapy device 310 rotating around the second axis may be greater than or equal to 90 degrees. In some embodiments, the rotation angle of the radiotherapy device 310 rotating around the second axis may be 100 degrees. In some embodiments, the rotation angle of the radiotherapy device 310 rotating around the second axis may be 120 degrees. In some embodiments, the rotation angle of the radiotherapy device 310 rotating around the second axis may be 150 degrees. In some embodiments, the rotation angle of the radiotherapy device 310 rotating around the second axis may be 180 degrees. In some embodiments, the rotation angle of the radiotherapy device 310 rotating around the second axis may be 210 degrees. In some embodiments, the rotation angle of the radiotherapy device 310 rotating around the second axis may be 240 degrees. In some embodiments, the rotation angle of the radiotherapy device 310 rotating around the second axis may be 270 degrees. In some embodiments, the rotation angle of the radiotherapy device 310 rotating around the second axis may be 300 degrees. In some embodiments, the rotation angle of the radiotherapy device 310 rotating around the second axis may be 330 degrees. In some embodiments, the rotation angle of the radiotherapy device 310 rotating around the second axis may be 360 degrees.

In some embodiments, the rotation angle of the radiotherapy device 310 rotating around the second axis may be less than 180 degrees. For example, in a reference coordinate system constructed with the extension direction of the table of the imaging device 320 as the X-axis, the rotation angle of the radiotherapy device 310 rotating around the Y-axis or the Z-axis may be less than 180 degrees.

In some embodiments of the present disclosure, the rotation angle of the radiotherapy device rotating around the second axis may be less than 180 degrees, allowing for a wider range of a treatment angle to be applied to the subject, ensuring a more comprehensive treatment on the subject.

In some embodiments, the rotation angle of the radiotherapy device 310 rotating around the second axis may be greater than or equal to 180 degrees. In some embodiments, the rotation angle of the radiotherapy device 310 rotating around the second axis may be 210 degrees. In some embodiments, the rotation angle of the radiotherapy device 310 rotating around the second axis may be greater than or equal to 240 degrees. In some embodiments, the rotation angle of the radiotherapy device 310 rotating around the second axis may be greater than or equal to 270 degrees. In some embodiments, the rotation angle of the radiotherapy device 310 rotating around the second axis may be greater than or equal to 300 degrees. In some embodiments, the rotation angle of the radiotherapy device 310 rotating around the second axis may be greater than or equal to 330 degrees. In some embodiments, the rotation angle of the radiotherapy device 310 rotating around the second axis may be greater than or equal to 360 degrees.

In some embodiments, when the imaging device includes the MRI device, and the radiotherapy device 310 rotates around the second axis, the radiotherapy device 310 may rotate in a range such that an angle between the direction of the radiation beam and the direction of the magnet field is within a range of 0°-45°. Additionally, the radiotherapy device 310 may move in a range and the direction of the radiation beam is perpendicular to the direction of the magnet field and an angle between the direction of the radiation beam and the Z-axis within a range of 0°-45°.

In some embodiments, when the imaging device includes the MRI device and the direction of the magnet field is perpendicular to the Z-axis and the X-axis (i.e., the direction of the magnet field is perpendicular to the plane defined by the X-axis and the Z-axis, and the plane is parallel to the coronal plane of the subject), and the radiotherapy device 310 may rotate around the Z-axis (i.e., the second axis) and the angle between the direction of the radiation beam of the radiotherapy device 310 and the direction of the magnet field within a range of 0°-45°, or a range of 5°-40°, or a range of 10°-35°, or a range of 15°-30°, or a range of 20°-25°.

In some embodiments, when the imaging device includes the MRI device and the direction of the magnet field is perpendicular to the Z-axis and the X-axis (i.e., the direction of the magnet field is perpendicular to the plane defined by the X-axis and the Z-axis and is parallel to the coronal plane of the subject), and the radiotherapy device 310 may rotate around the Y-axis (i.e., the second axis), and the direction of the radiation beam perpendicular to the direction of the magnet field and the angle between the direction of the radiation beam of the radiotherapy device 310 and the direction of the magnet field within a range of 0°-45°, or a range of 5°-40°, or a range of 10°-35°, or a range of 15°-30°, or a range of 20°-25°.

In some embodiments, when the imaging device includes the MRI device and the direction of the magnet field is parallel to the X-axis (i.e., the direction of the magnet field is perpendicular to the plane defined by the X-axis and the Z-axis, and parallel to the traverse section of the subject), the radiotherapy device 310 may rotate around the Z-axis (i.e., the second axis) or the Y-axis (i.e., the second axis), and the angle between the direction of the radiation beam of the radiotherapy device 310 and the direction of the magnet field within a range of 0°-45°, or a range of 5°-40°, or a range of 10°-35°, or a range of 15°-30°, or a range of 20°-25°.

The imaging device 320 may be configured to image the subject (e.g., the head of the patient). In some embodiments, the imaging device 320 may be the MRI device. The MRI device may be configured to perform magnetic resonance imaging on the subject (e.g., the head of the patient). The magnetic resonance imaging may be a process of forming an image by using hydrogen atomic nuclei in human tissues in a static magnetic field to undergo magnetic resonance phenomena when stimulated by radio frequency pulses of a specific frequency. It may display clear and fine images of the lesion site from a plurality of angles and planes to assist doctors in diagnosis.

In some embodiments, the magnetic resonance imaging device may include a magnet assembly and a gantry.

The gantry may be a structure for supporting the magnet assembly. The magnet assembly may be provided on the gantry. In some embodiments, the gantry may include a C-shaped structure, a frame structure, or the like. The material of the gantry may include a metal material, an alloy material, or the like, or a combination thereof.

The magnet assembly may be configured to generate the magnet field in the accommodation space. The accommodation space refers to a space in the magnetic resonance imaging device for accommodating the subject. The magnet assembly may include at least one magnet.

In some embodiments, the magnet assembly may include a permanent magnet, an electromagnet, or the like, or a combination thereof. The permanent magnet, as the magnet assembly, may maintain magnetism for a long period, and have a low operating cost. However, the permanent magnet has a low magnet field strength, difficulty in debugging the uniformity of the magnet field, and high requirements for the installation environment. The electromagnet, as the magnet assembly, may have a high magnet field strength, and a better magnet field uniformity, but the running cost is higher.

In some embodiments, the magnet assembly may be an open magnet. The open magnet may be composed of two open magnet portions, with a magnetic field distribution formed between the two magnet portions and a space for accommodating the subject. For example, the magnet assembly may include an open permanent magnet or an open electromagnet. A gamma knife system guided by the open magnet (e.g., the permanent magnet or the electromagnet) may reduce the oppression of the treatment process for the subject (e.g., the patient), which may be friendly to people with some medical conditions (e.g., claustrophobia).

In some embodiments, the magnet assembly may be a cylindrical magnet such as a horizontal cylindrical magnet. An interior of the cylindrical magnet may have an accommodation space, and the subject may lie in the accommodation space along the extension direction of the table. The magnet assembly may generate the magnet field with a horizontal direction (i.e., parallel to the X-axis) within the accommodation space.

In some embodiments, the magnet assembly may include a first magnet and a second magnet. The first magnet may be provided on a side of the accommodation space in a direction parallel to the direction of the magnet field, and the second magnet may be provided on another side of the accommodation space in a direction parallel to the direction of the magnet field. The first magnet may cooperate with the second magnet to form the magnet field.

In some embodiments, the accommodation space between the first magnet and the second magnet may be configured to accommodate the subject. In some embodiments, the radiotherapy device 310 may emit rays toward the accommodation space between the first magnet and the second magnet to perform radiation treatment on the subject.

In some embodiments of the present disclosure, utilizing the gamma knife system guided by the open magnet may reduce the oppressive sense of the treatment process for the subject (e.g., the patient), which may be friendly to people with some medical conditions (e.g., claustrophobia).

In some embodiments, when the imaging device includes the MRI device, a correspondence between the direction of the radiation beam and the direction of the magnet field may be involved.

In some embodiments, when the imaging device include the MRI device and the magnet assembly include the first magnet and the second magnet, the direction of the radiation beam emitted by the radiotherapy device 310 may be parallel or approximately parallel to the direction of the magnet field. For example, a relative position of the radiotherapy device 310 to the imaging device 320 may be adjusted such that the direction of the radiation beam emitted by the radiotherapy device 310 may be parallel or approximately parallel to the direction of the magnet field. As used herein, the direction of the radiation beam emitted by the radiotherapy device 310 approximately parallel to the direction of the magnet field refers to that an angle between the direction of the radiation beam emitted by the radiotherapy device 310 and the direction of the magnet field is less than a threshold, such as 5 degrees, 10 degrees, 15 degrees, 20 degrees, 30 degrees, etc. When the direction of the radiation beam emitted by the radiotherapy device 310 is parallel to the direction of the magnet field, the magnet field may produce less effect on the radiation beam. More descriptions regarding the direction of the radiation beam emitted by the radiotherapy device 310 being parallel or approximately parallel to the direction of the magnet field may be found in FIG. 4A and related descriptions thereof.

In some embodiments of the present disclosure, the direction of the radiation beam may be parallel or approximately parallel to the direction of the magnet field, reducing the attenuation of the radiation beam, which in turn leads to better online radiation treatment.

In some embodiments, the direction of the radiation beam emitted by the radiotherapy device 310 may be perpendicular or approximately perpendicular to the direction of the magnet field when the magnet assembly includes the first magnet and the second magnet. For example, the relative position of the radiotherapy device 310 to the imaging device 320 may be adjusted such that the direction of the radiation beam emitted by the radiotherapy device 310 may be perpendicular or approximately perpendicular to the direction of the magnet field. More descriptions regarding the direction of the radiation beam emitted by the radiotherapy device 310 being perpendicular or approximately perpendicular to the direction of the magnet field may be found in FIG. 4B and related descriptions thereof.

In some embodiments of the present disclosure, when the direction of the radiation beam is perpendicular or approximately perpendicular to the direction of the magnet field, the radiation beam emitted by the radiotherapy device 310 may not need to pass through the gantry or the magnet assembly, which may avoid the attenuation of the radiation beam and allow the magnet field between the first magnet and the second magnet to be more uniform, thereby allowing for better online magnetic resonance imaging and online radiation treatment on the subject.

In some embodiments, when the magnet assembly includes the first magnet and the second magnet, at least one rotation axis may include a first axis perpendicular to the direction of the magnet field in the accommodation space and a second axis perpendicular or parallel to the direction of the magnet field in the accommodation space. For example, in a reference coordinate system constructed with the extension direction of the table of the imaging device 320 as the X-axis, the first axis may be a straight line parallel to the X-axis and the second axis may be a straight line parallel to the Y-axis or the Z-axis.

In some embodiments of the present disclosure, the rotation axis may be perpendicular or parallel to the direction of the magnet field such that when the radiotherapy device rotates around the rotation axis, the angle between the direction of the magnet field and the direction of the radiation beam may be more clearly defined, which may improve the precision of the radiation treatment.

In some embodiments, an upper portion of the gantry of the imaging device 320 may be provided with a guiding slot. The upper portion of the gantry may be a side of the gantry that is higher than other portions of the gantry.

The guiding slot may be configured for the radiation beam to pass through, i.e., the radiation beam emitted by the radiotherapy device 310 may be irradiated on the subject through the guiding slot. In some embodiments, the guiding slot may be a slit having a certain width, and the radiation beam of the radiotherapy device may rotate around the second axis within a range of the guiding slot (e.g., a range of the width of the slit). In some embodiments, the gantry may include another guiding slot and the guiding slot may be an annular structure perpendicular to the first axis. The radiation beam of the radiotherapy device may rotate around the first axis within a range of the guiding slot (e.g., a range of the width of the slit).

In some embodiments, the guiding slot at the upper portion of a gantry of the magnetic resonance imaging device may not be necessary, and the radiation beam of the radiotherapy device may pass directly through the magnet assembly.

In some embodiments of the present disclosure, when the guiding slot is provided at the upper portion of the gantry of the MRI device, the radiation beam may pass through the magnet assembly through the guiding slot, which may effectively avoid the attenuation of the intensity of the radiation beam.

In some embodiments, due to the guiding slot being provided on the upper portion of the gantry of the imaging device 320, an optimized design (e.g., increasing the volume of the first magnet, or a localized optimized design, etc.) may be adopted for the magnet (e.g., the first magnet close to the guiding slot) at the end where the guiding slot is located to ensure the uniformity of the magnet field between the first magnet and the second magnet.

In some embodiments, due to the guiding slot being provided on the upper portion of the gantry of the imaging device 320, and/or a recess is provided on each of the first magnet and the second magnet which are configured to pass the radiation beam.

The recess may be configured for the radiation beam to pass through, i.e., the radiation beam emitted by the radiotherapy device 310 may be irradiated on the subject through the recess.

In some embodiments, the recess at each of the first magnet and the second magnet may not be necessary, and the radiation beam of the radiotherapy device may pass directly through the magnet assembly.

In some embodiments of the present disclosure, when the guiding slot is provided at the upper portion of the gantry of the MRI device, and/or the recesses are provided in the first magnet and the second magnet, the radiation beam may pass through the magnet assembly through the guiding slot and the recesses, which may effectively avoid the attenuation of the intensity of the radiation beam and reduce the loss of the radiation beam.

In some embodiments, when the radiotherapy device 310 emits a radiation beam that passes through the guiding slot or directly through the magnet assembly, if the radiotherapy device 310 is a linear gas pedal, the magnet field may have less effect on the radiation beam when the radiotherapy device 310 emits a radiation beam having a direction parallel to the direction of the magnet field.

In some embodiments, the magnet assembly may include one or more magnets, and the one or more magnets may all be a pipe structure, which may be provided around the accommodation space.

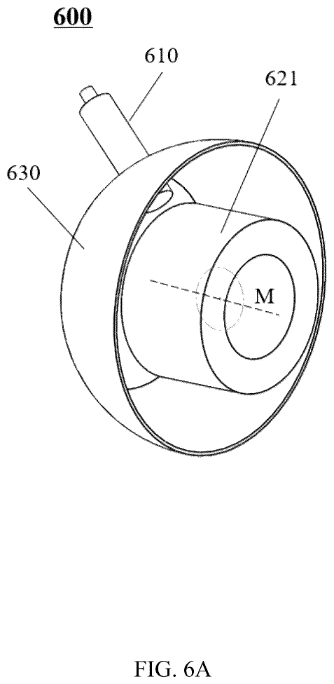

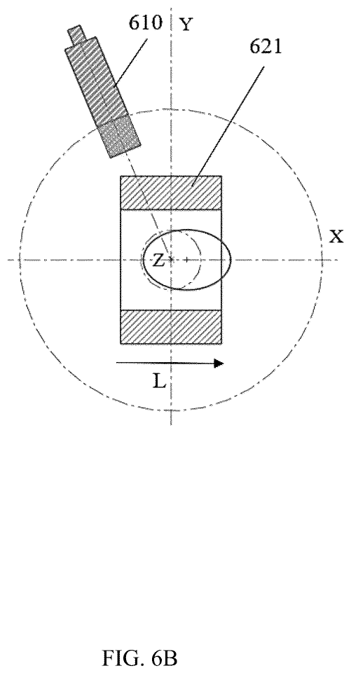

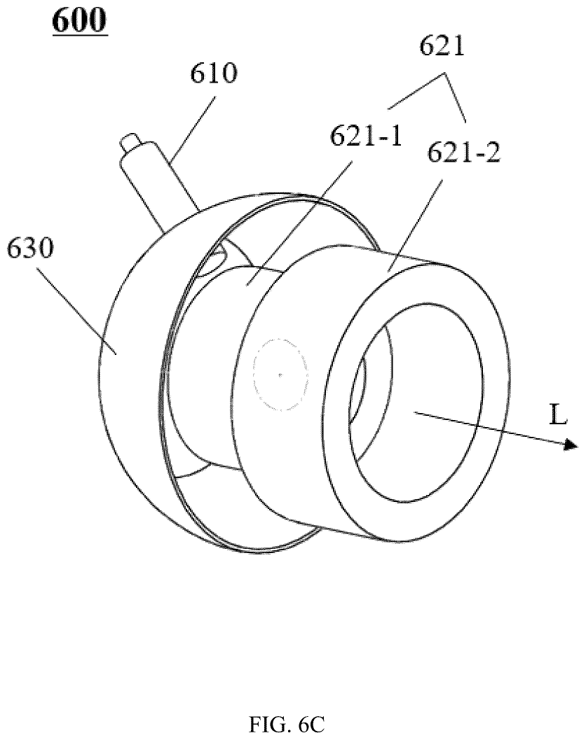

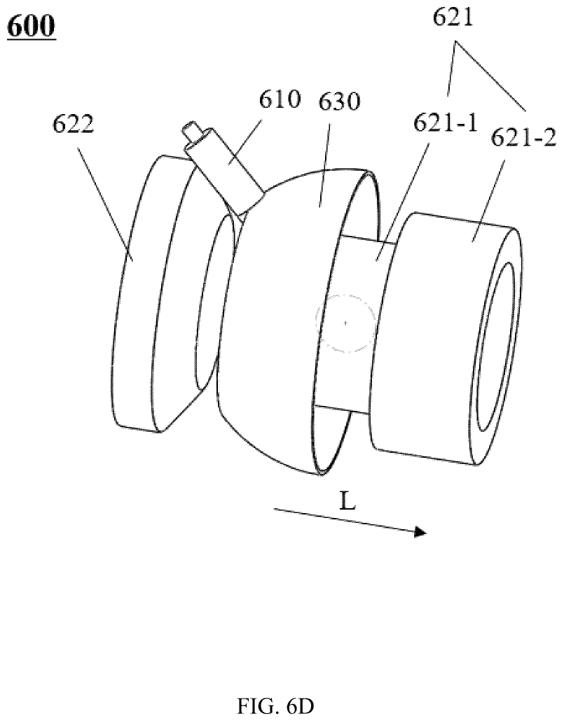

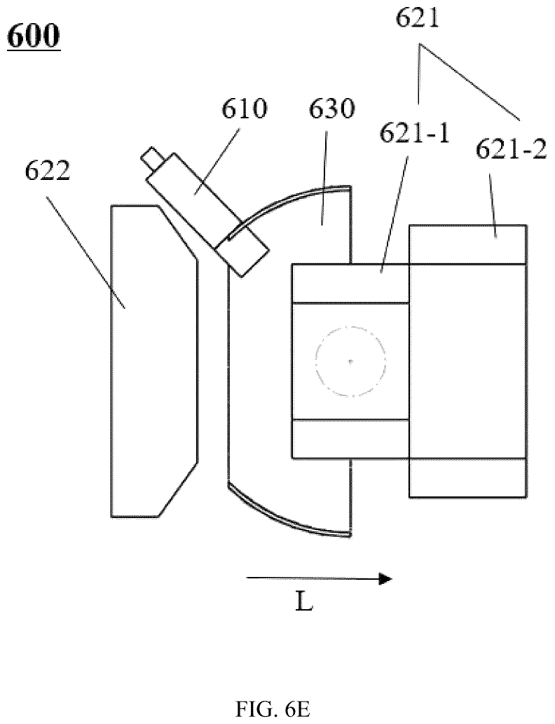

In some embodiments, when the magnet assembly includes a magnet, the magnet may be of the pipe structure. For example, the magnet may be a cylindrical magnet. The cylindrical magnet may have a certain accommodation space inside the cylindrical magnet, and the direction of the magnet field may be formed in the accommodation space parallel to the central axis of the cylindrical magnet along the center of an aperture of the cylindrical magnet. More descriptions regarding this embodiment may be found in FIG. 6A and related descriptions thereof.

In some embodiments, when the magnet assembly includes a plurality of magnets, the plurality of magnets may be of the pipe structure. The plurality of magnets of the pipe structures may be connected in series. For example, the plurality of magnets may be cylindrical magnets of the same diameter, with two ends sequentially connected to form a large pipe structure. More descriptions regarding this embodiment may be found in FIG. 6C-FIG. 6E and related descriptions thereof.

In some embodiments of the present disclosure, one or more magnets of the pipe structure (e.g., the cylindrical magnet) may be provided around the accommodation space, and the cylindrical magnet with the magnet field with greater uniformity may be formed.

In some embodiments, when the magnet assembly includes one or more magnets that form the pipe structure, the at least one rotation axis may include a first axis parallel to the direction of the magnet field in the accommodation space and a second axis perpendicular to the direction of the magnet field in the accommodation space. For example, in a reference coordinate system constructed with the extension direction of the table of the imaging device 320 as the X-axis, etc., the first axis may be a straight line parallel to the X-axis and the second axis may be a straight line parallel to the Y-axis or the Z-axis.

In some embodiments of the present disclosure, when the magnet assembly is the cylindrical magnet and the rotation axis includes the first axis parallel to the direction of the magnet field in the accommodation space and the second axis perpendicular to the direction of the magnet field in the accommodation space, the radiotherapy device may rotate around the first axis and the second axis, and a precise online radiation treatment may be realized based on the structure of the cylindrical magnet.

In some embodiments, when the magnet assembly includes one or more magnets forming the pipe structure, the magnet assembly may include a first section (e.g., a cylindrical section) and a second section (e.g., a cylindrical section), and a size of the first section may be smaller than a size of the second section, wherein the size indicates the diameter. The first section may be used to accommodate the patient's head and the second section may be used to accommodate portions of the patient other than the head, such as the shoulders.

Each cylindrical segment may include one or more magnets. In some embodiments, the first section and the second section may be provided sequentially around the accommodation space along the direction parallel to the central axis. The first section may be farther to the entrance of the treatment apparatus where the table enters the accommodating space from than the second section. The second section and the first section may be fixedly connected.

In some embodiments, the first section may be configured to accommodate the head of a human body and the second section may be configured to accommodate the shoulder of the human body. In some embodiments, a ratio of the diameter of the first section to the diameter of the second section and/or a ratio of the length of the first section to a length of the second section may be set to be different to adapt to human bodies of different head and body sizes.

In some embodiments, compared to the magnet assembly including a whole cylindrical magnet (as shown in FIG. 6A), the magnet assembly including multiple segment magnets (e.g., the first cylindrical magnet and the second cylindrical magnet as shown in FIG. 6C) may have a total length of greater than the length of the whole cylindrical magnet, which may improve the uniformity of the magnet field at the multiple segment magnets to facilitate better magnetic resonance imaging of the head of the patient.

More descriptions regarding this embodiment may be found in FIG. 6C and related descriptions thereof.

In some embodiments of the present disclosure, the magnet assembly may include the first section and the second section, and the diameter of the first section may be smaller than the diameter of the second section, which may result in a higher magnetic density inside the first section, and a better magnetic resonance imaging effect.

In some embodiments, when the magnet assembly includes one or more magnets each of which is in a tubular structure (also referred as cylindrical magnet) and the third magnet, the magnet assembly may include the first section and the second section. Thus, the cylindrical magnet and the third magnet may be provided on two sides of the radiotherapy device and the support along the direction of the magnet field, and the cylindrical magnet may be configured to form the accommodation space.

In some embodiments, the first magnet may be provided on a side toward the direction of the radiation beam of the radiotherapy device 310, and the second magnet may be provided on a side away from the direction of the radiation beam of the radiotherapy device 310. In this embodiment, the first magnet may include one or more magnets, and the one or more magnets may all be of the pipe structure, which may be provided around the accommodation space. The first magnet may further include the first section and the second section, and the diameter of the first section may be smaller than diameter of the second section.

More descriptions regarding this embodiment may be found in FIG. 6E and related descriptions thereof.

In some embodiments of the present disclosure, by adding the second magnet on the side away from the direction of the radiation beam of the radiotherapy device, the magnetic density inside the first magnet may be made higher and the uniformity of the magnet field may be made better, to make the magnetic resonance imaging effect to be better.

The support 330 may be configured to provide a support for the imaging device 320 and the radiotherapy device 310. For example, the support 330 may be a stand, etc.

In some embodiments, the radiotherapy device 310 and the support 330 may be fixedly connected. In some embodiments, the support 330 may drive the radiotherapy device 310 to achieve rotation.

In some embodiments, the support 330 may include a rotating shell. In some embodiments, the rotating shell may be a shell of a shape, e.g., sphere, hemisphere, etc. In some embodiments, the rotating shell may be in a semicircular shape. In some embodiments, the rotating shell in a semicircular shape may be configured to provide the support to the radiotherapy device. In some embodiments, the support (e.g., the rotating shell) may be configured to restrict part of the radiation beam emitted outside the accommodation space and/or the shell, avoiding the existence of the radiation beam outside the accommodation space and/or the space defined by the shell.

In some embodiments, the treatment system 110 may include a shielding structure (e.g., a magnetic shielding material not shown in FIG. 4B) to minimize the effect of the magnet field on a radiation beam.

In some embodiments of the present disclosure, by combining the MRI device and the radiotherapy device into an integrated device, the magnetic resonance imaging device and the radiotherapy device may be interlinked and controlled, and the radiotherapy device may rotate around the rotation axis. In such a case, an online three-dimensional angular treatment may be performed on the subject, making the treatment on the subject more accurate. Further, the use of different ray doses at different angles between the direction of the radiation beam and the direction of the magnet field may reduce damage to other parts of the body (e.g., the mouth, the nose, etc.). The treatment system may be manufactured using the permanent magnet, the electromagnet, etc., and using a plurality of structural forms such as an open magnet, a horizontal cylindrical magnet, etc., which may be adapted to different application scenarios.

FIG. 4A is a schematic diagram illustrating an exemplary gamma knife system according to some embodiments of the present disclosure. FIG. 4B is a schematic diagram illustrating a section view of the gamma knife system as shown in FIG. 4A t according to some embodiments of the present disclosure. Some of the following embodiments may be understood with reference to FIGS. 4A and 4B, but the accompanying drawings are only schematic illustrations of some of these embodiments and do not constitute a limitation of the embodiments.

In some embodiments, as shown in FIGS. 4A and 4B, the gamma knife system 400 may include a radiotherapy device 410, a magnetic resonance imaging device 420, and a support. In this embodiment, the support may include a rotating shell 430.

In some embodiments, the magnetic resonance imaging device 420 may include a magnet assembly 421 and a gantry 422, and the magnet assembly 421 may be provided on the gantry 422. As shown in FIGS. 4A and 4B, the gantry 422 may be of a C-shaped structure. In some embodiments, the radiotherapy device 410 may be a linear gas pedal. In some embodiments, a direction of a radiation beam of the radiotherapy device 410 may be parallel or approximately parallel to a direction of a magnet field of the magnet assembly 421, i.e., the direction of the radiation beam of the radiotherapy device 410 may be parallel or approximately parallel to the direction shown by H in FIG. 4A.

In some embodiments, a three-dimensional reference coordinate system may be predetermined in the gamma knife system 400 shown in FIGS. 4A and 4B. For example, an axis perpendicular to the direction H of the magnet field and parallel to an axis M of the rotating shell may be an X-axis (e.g., a direction parallel to a paper surface pointing to the right may be a positive direction of the X-axis), an axis parallel to the direction H of the magnet field may be a Y-axis (e.g., a direction parallel to the paper surface pointing to the up may be a positive direction of the Y-axis), and an axis perpendicular to the direction H of the magnet field and perpendicular to the axis M of the rotating shell may be a Z-axis (e.g., a direction perpendicular to the paper surface pointing to the up may be preset to be a positive direction of the Z-axis).

In some embodiments, the radiotherapy device 410 may rotate around at least one rotation axis. In some embodiments, the at least one rotation axis may be perpendicular and/or parallel to the direction of the magnet field of the magnet. For example, the at least one axis may be perpendicular to the direction of the magnet field in FIG. 4A (the direction shown by H).

In some embodiments, the at least one axis may include the X-axis shown in FIG. 4B. In some embodiments, the at least one axis may also include the Z-axis as shown in FIG. 4B.

In some embodiments, the radiotherapy device 410 may rotate around the X-axis as shown in FIG. 4B. In some embodiments, a rotation angle of the radiotherapy device 410 rotating around the X-axis shown in FIG. 4B may be within a range of 0°-360°. In some embodiments, the magnetic resonance imaging device 420 may rotate synchronously with a rotation of the radiotherapy device 410 when the radiotherapy device 410 rotates around the X-axis as shown in FIG. 4B, to minimize attenuation of treatment radiation rays.

In some embodiments, when the radiotherapy device 410 rotates around the Z-axis shown in FIG. 4B, a treatment angle in a sagittal plane (i.e., a plane in which the X-axis and the Y-axis are located) of the subject may be less than 90°, which may reduce injury to organs in other parts of the body outside of the subject (e.g., the mouth, the nose, etc.) to achieve a more precise treatment on the subject.

In some embodiments, the radiotherapy device 410 may rotate around the at least one rotation axis and an angle between the direction of the radiation beam and the direction of the magnet field (i.e., the direction shown by H in FIG. 4A) within a range of 0°-45°. In some embodiments, if the MRI device 420 rotates synchronously with the rotation of the radiotherapy device 410 when the radiotherapy device 410 rotates around the X-axis shown in FIG. 4A, the angle between the direction of the radiation beam and the direction of the magnet field of the radiotherapy device 410 may be considered to remain constant. When the radiotherapy device 410 rotates around the Z-axis shown in FIG. 4B, the angle between the direction of the radiation beam and the direction of the magnet field within a range of 0°-45°. In some embodiments, when the radiotherapy device 410 rotates around the Z-axis shown in FIG. 4B, the angle between the direction of the radiation beam and the direction of the magnet field within a range of 5°-40°. In some embodiments, when the radiotherapy device 410 rotates around the Z-axis shown in FIG. 4B, the angle between the direction of the radiation beam and the direction of the magnet field within a range of 10°-35°. In some embodiments, when the radiotherapy device 410 rotates around the Z-axis shown in FIG. 4B, the angle between the direction of the radiation beam and the direction of the magnet field within a range of 15°-30°. In some embodiments, when the radiotherapy device 410 rotates around the Z-axis shown in FIG. 4B, the angle between the direction of the radiation beam and the direction of the magnet field within a range of 20°-25°.

In some embodiments, according to the needs of actual treatment, different ray doses may be used when the angle between the direction of the radiation beam of the radiotherapy device 410 and the direction of the magnet field is different. For example, when the angle between the direction of the radiation beam of the radiotherapy device 410 and the direction of the magnet field is A1, a ray dose of the radiation beam emitted by the radiotherapy device 410 may be B1. When the angle between the direction of the radiation beam of the radiotherapy device 410 and the direction of the magnet field is A2, a ray dose of B2 may be used.

In some embodiments of the present disclosure, by setting the radiotherapy device 410 to rotate around the Z-axis and/or the X-axis shown in FIG. 4B and the angle between the direction of the radiation beam of the radiotherapy device 410 and the direction of the magnet field of the magnet assembly 421 to be acute, the treatment angle of the subject may be three-dimensional, and the treatment angle may be 360° on the treatment in a traverse section of the subject. The MRI device 420 may rotate synchronously with the rotation of the radiotherapy device 410 to reduce the attenuation of the treatment radiation rays. The treatment angle may be less than 90° on the sagittal plane of the subject, and different ray doses may be used when the angle between the direction of the radiation beam and the direction of the magnet field is different. Therefore, damages to other parts of the organs outside of the subject (e.g., the mouth, the nose, etc.) may be reduced, thereby allowing for a more precise treatment on the subject.

In some embodiments, the magnet assembly 421 may include the magnet a first magnet 421-1 and a second magnet 421-2, and the first magnet 421-1 and the second magnet 421-2 may be provided on two ends of the gantry 422 along the direction of the magnetic field. As shown in FIG. 4A, the first magnet 421-1 and the second magnet 421-2 may be provided inside an upper portion and a lower end of a C-shaped structure of the gantry 422, respectively. The first magnet 421-1 and the second magnet 421-2 may form a magnet field in a vertical direction, i.e., the direction shown by H may be the direction of the magnet field. In some embodiments, an opening accommodation space may be formed between the first magnet 421-1 and the second magnet 421-2 for accommodating the subject. The magnet assembly 421 may also be referred to as an opening magnet assembly. The magnetic resonance imaging device 420 may also referred to as an opening MRI device.

In some embodiments, to ensure that the radiation beam of the radiotherapy device 410 may be irradiated on the subject, a guiding slot 422-1 may be provided at one end (e.g., the upper portion) of the gantry 422, recesses 423 may be provided at the first magnet 421-1 and the second magnet 421-2, and the radiation beam of the radiotherapy device 410 may be irradiated on the subject through the guiding slot 422-1. In some embodiments, the guiding slot 422-1 may be a slit having a certain width, and the radiation beam of the radiotherapy device 410 may rotate within a range of the slit of the guiding slot 422-1. In some embodiments, since the guiding slot 422-1 is provided at one end (e.g., the upper portion) of the gantry 422, to ensure the uniformity of the magnet field between the first magnet 421-1 and the second magnet 421-2, an optimized design (e.g., increasing a volume of the first magnet 421-1 or a localized optimized) may be performed on the magnet on an end where the guiding slot 422-1 (e.g., the first magnet 421-1) is located. In some embodiments, when the gantry 422 is provided with the guiding slot 422-1, the radiation beam of the radiotherapy device 410 may pass through the magnet 4421 through the guiding slot 422-1, which may effectively prevent attenuation of the intensity of the radiation beam. In some embodiments, the guiding slots 422-1 may not be necessary to be provided on the gantry 422, and the radiation beam of the radiotherapy device 410 may pass directly through the magnet assembly 421. In some embodiments, when the radiation beam of the radiotherapy device 410 passes through the guiding slot 422-1 or directly through the magnet assembly 421, if the radiotherapy device 410 is a linear gas pedal, the magnet field may produce less effect on the radiation beam of the radiotherapy device 410 when the direction of the radiation beam of the radiotherapy device 410 is parallel, or approximately parallel, to the direction of the magnet field.

In some embodiments, the radiotherapy device 410 may emit radiation rays toward the accommodation space between the first magnet 421-1 and the second magnet 421-2 to perform the radiation treatment on the subject. In some embodiments, the angle between the direction of the radiation beam of the radiotherapy device 410 and the direction of the magnet field of the magnet assembly 421 (i.e., the direction shown by H) may be acute. In some embodiments, since the rays emitted by the radiotherapy device 410 need to pass through the magnet assembly 421, the rotation angle of the radiotherapy device 410 rotating around the X-axis may be 360° on the traverse section of the subject (i.e., on the plane where the Y-axis and Z-axis are located).

In some embodiments, the rotating shell 430 may be a hemispherical rotating shell, as shown in FIG. 4A. In some embodiments, the radiotherapy device 410 may be arranged on the rotating shell 430. The rotating shell 430 may provide a support to the radiotherapy device 410. The radiotherapy device 410 may rotate along the rotation of the rotating shell 430. Therefore, a treatment at a three-dimensional angle may be performed on the subject, resulting in a better treatment effect.

By combining the MRI device 420 and the radiotherapy device 410 into an integrated device, the MRI device 420 and the radiotherapy device 410 may be interlinked and controlled, and a total size of the device may be reduced. According to the magnetic resonance imaging of the magnetic resonance imaging device 420, an online localization of the subject may be achieved. The radiotherapy device 410 may perform online treatment on the subject based on a magnetic resonance image, realizing online three-dimensional angular treatment on the subject, i.e., treatment at an angle of 360° on the traverse section and treatment at an angle of less than 90° on the sagittal plane. Further, the magnetic resonance imaging device 420 may rotate synchronously with the rotation of the radiotherapy device 410, reducing the attenuation of the treatment radiation rays, and reducing damages to other parts of the organs outside of the subject (e.g., the mouth, the nose, etc.), thereby allowing for a more precise treatment on the subject.

FIG. 4C is a schematic diagram illustrating another exemplary gamma knife system according to some embodiments of the present disclosure.

The gamma knife system 450 shown in FIG. 4C is the same as or similar to the gamma knife system guided by the open permanent magnet 400 shown in FIG. 4A. For example, the gamma knife system 450 shown in FIG. 4C may include a support, a radiotherapy device 410, and a magnetic resonance imaging (MRI) device 420. The support is not shown in FIG. 4C.

Unlike the gamma knife system guided by the open permanent magnet 400, a location of setting the radiotherapy device 410 in the gamma knife system guided by magnetic resonance imaging 450 is different. That is, the radiotherapy device 410 in the gamma knife system 450 is located on a side of the imaging device 420, and the radiotherapy device 410 in the gamma knife system 400 is located on an upper portion of the MRI device 420.

In some embodiments, when the imaging device includes a magnetic resonance imaging (MRI) device, as shown in FIG. 4C, a direction of a radiation beam of the radiotherapy device 410 may be perpendicular or approximately perpendicular to a direction of a magnet field of the magnet assembly 421, i.e., the direction of the radiation beam of the radiotherapy device 410 may be perpendicular or approximately perpendicular to the direction shown by H in FIG. 4C.

In some embodiments, the radiotherapy device 410 may rotate around at least one rotation axis. In some embodiments, as shown in FIG. 4C, the at least one rotation axis may include a rotation axis perpendicular and/or a rotation axis parallel to the direction of the magnet field of the magnet assembly 421 (i.e., the direction shown by H in FIG. 4C).

In some embodiments, the rotation axis may include at least an X-axis perpendicular to a traverse section of a subject. In some embodiments, a rotation angle of the radiotherapy device 410 rotating around the X-axis shown in FIG. 4D may be 360° in a traverse section of the subject (i.e., a plane in which a Y-axis and a Z-axis are located). In some embodiments, the MRI device 420 may rotate synchronously with a rotation of the radiotherapy device 410 as the radiotherapy device 410 rotates around the X-axis to reduce attenuation of treatment radiation rays.

In some embodiments, the at least one rotation axis may also include a Y-axis parallel to the direction of the magnet field of the magnet assembly 421 (i.e., the direction shown by H in FIG. 4C). In some embodiments, the radiotherapy device 410 may rotate around the Y-axis shown in FIG. 4C. In some embodiments, the radiotherapy device 410, when rotates around the Y-axis shown in FIG. 4C, always rotates in a plane perpendicular to the Y-axis shown in FIG. 4C (i.e., the plane in which the X-axis and Z-axis are located as shown in FIG. 4C). The rotation angle may be less than 90 degrees, such that the treatment angle may be less than 90°, which reduces damage to other parts of the organs outside of the subject (e.g., the mouth, the nose, etc.), and thus perform a more precise treatment on the subject.

It should be noted that since the MRI device 420 rotates synchronously with the rotation of the radiotherapy device 410 when the radiotherapy device 410 rotates around the X-axis shown in FIG. 4C, an angle between the direction of the radiation beam of the radiotherapy device 410 and the direction of the magnet field may be considered to remain constant.

In some embodiments, the radiotherapy device 410 may rotate around the Y axis and the direction of the radiation beam is perpendicular to the direction of the magnet field and at an angle between the direction of the radiation beam and the Z-axis as shown in FIG. 4C within a range of 0°-45°.

In some embodiments, when the radiotherapy device 410 rotates around the Y-axis shown in FIG. 4C, the direction of the radiation beam is perpendicular to the direction shown by H in FIG. 4C and an angle between the direction of the radiation beam and the Z-axis as shown in FIG. 4C within a range of 5°-45°, or a range of 10°-35°, or a range of 20°-30°.

More descriptions regarding the gamma knife system guided by the open permanent magnet 400 may be found in the detailed descriptions in FIG. 4A, which may not be repeated here.

As shown in FIG. 4C, by setting the direction of the radiation beam of the radiotherapy device 410 to be perpendicular to the direction of the magnet field of the magnet assembly 421, the treatment radiation rays emitted by the radiotherapy device 410 may be unnecessary to pass through the gantry 422 or the magnet assembly 421. Thus, attenuation of the treatment radiation rays may not occur, and a guiding slot may not be required to be provided on the gantry 422, thereby reducing the manufacturing difficulty of the magnetic resonance imaging device 420 and reducing the manufacturing process. Furthermore, the magnet field between the first magnet 421-1 and the second magnet 421-2 may be uniform, which allows for better online magnetic resonance imaging and online radiation treatment on the subject.

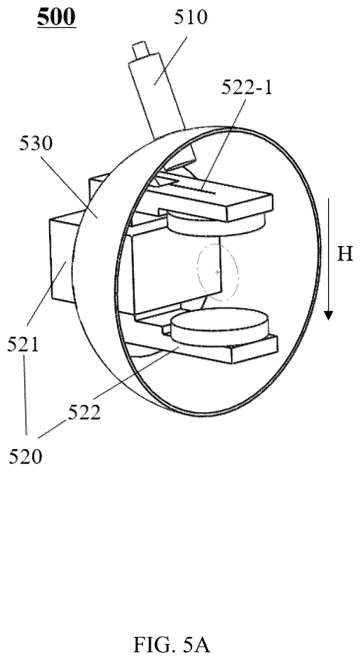

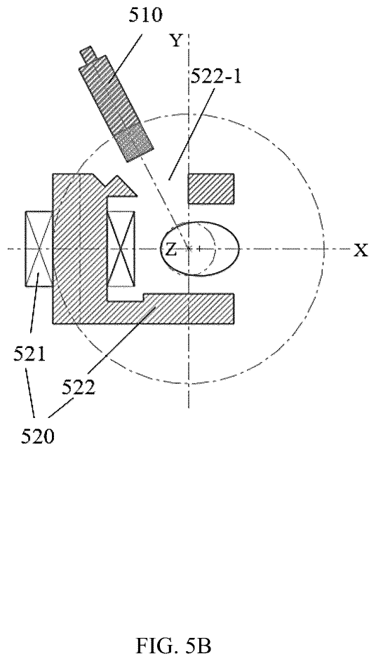

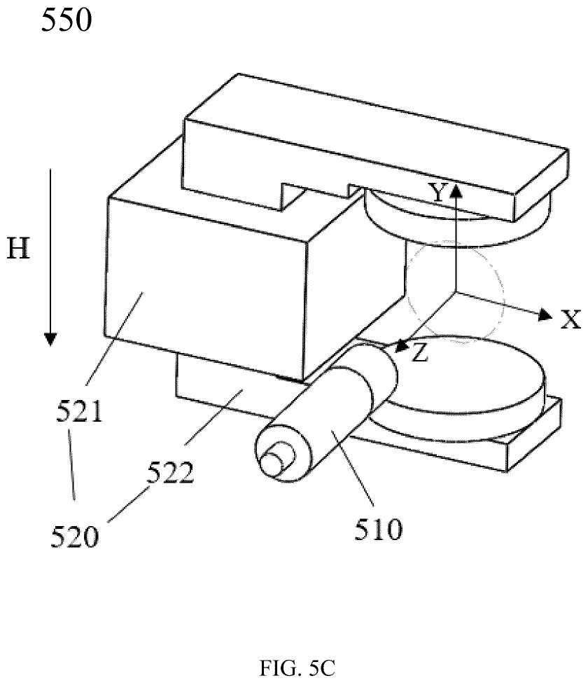

FIG. 5A is a schematic diagram illustrating an exemplary gamma knife system according to some embodiments of the present disclosure. FIG. 5B is a schematic diagram illustrating a section view of the gamma knife system as shown in FIG. 5A according to some embodiments of the present disclosure. The gamma knife system 500 shown in FIGS. 5A and 5B is similar in structure to the gamma knife system 400 shown in FIG. 4A, and mainly different in the type of magnet.

Some of the following embodiments may be understood with reference to FIGS. 5A and 5B, but the accompanying drawings are only illustrative of some of these embodiments and do not constitute a limitation of the embodiments.

In some embodiments, the gamma knife system 500 may include a radiotherapy device 510, a magnetic resonance imaging (MRI) device 520, and a support, as shown in FIG. 5A. In this embodiment, the support may include a rotating shell 530.

In some embodiments, the magnetic resonance imaging device 520 may include a magnet assembly 521 and a gantry 522, with the magnet assembly 521 provided on the gantry 522.

In some embodiments, the radiotherapy device 510 may rotate around at least one rotation axis. In some embodiments, the at least one rotation axis may include a rotation axis perpendicular to a direction of a magnet field of a magnet. For example, the rotation axis may be perpendicular to the direction of the magnet field in FIG. 5A (the direction shown by H in FIG. 5A). In some embodiments, a direction of a radiation beam of the radiotherapy device 510 may be parallel or approximately parallel to the direction of the magnet field of the magnet assembly 521, i.e., the direction of the radiation beam of the radiotherapy device 510 may be parallel or approximately parallel to the direction shown by H in FIG. 5A.

In some embodiments, a three-dimensional reference coordinate system may be preset in the gamma knife system 500 shown in FIG. 5B. The three-dimensional coordinate system may be preset in this embodiment in a manner consistent with that in FIG. 4B. More descriptions may be found in related descriptions in the preceding section.

In some embodiments, as shown in FIG. 5B, the at least one rotation axis may include at least an X-axis perpendicular to a traverse section of a subject, and the X-axis may be an axis parallel to an axis of the rotating shell 530 and perpendicular to the direction of the magnet field. In some embodiments, when the radiotherapy device 510 rotates around the X-axis shown in FIG. 5B, a treatment angle may be 360° in the traverse section of the subject. In some embodiments, when the radiotherapy device 510 rotates around the X-axis shown in FIG. 5B, the magnetic resonance imaging device 520 may rotate synchronously with the rotation of the radiotherapy device 510 to reduce the attenuation of the treatment radiation rays. In some embodiments, as shown in FIG. 5B, the at least one rotation axis may further include a Z-axis, and the Z-axis may be perpendicular to the axis of the rotating shell 530 and perpendicular to the direction of the magnet field.

In some embodiments, the magnet assembly 521 may include an electromagnet, as shown in FIG. 5A. In some embodiments, the electromagnet may include one or more coils provided on the gantry 522. In some embodiments, the electromagnet may include coils provided in the middle of a C-shaped structure of the gantry 522, as shown in FIG. 5A.

In some embodiments, to ensure that the radiation beam of the radiotherapy device 510 may be irradiated on the subject, a guiding slot 522-1 may be provided at one end (e.g., an upper portion) of the gantry 522, and the radiation beam of the radiotherapy device 510 may be irradiated on the subject through the guiding slot 522-1. More descriptions regarding the radiotherapy device 510 may be found in the detailed description of the radiotherapy device 410 in FIG. 4A, which may not be repeated here.

In some embodiments, the radiotherapy device 510 and the rotating shell 530 may be fixedly provided, the rotating shell 530 may provide a support to the radiotherapy device 510, and the radiotherapy device 510 may be driven by the rotating shell 530 to rotate, to realize a three-dimensional angular treatment on the subject, and make the treatment effect better.

More descriptions regarding the gamma knife system 500 may be found in the related description of FIG. 4A, which may not be repeated here.

FIG. 5C is a schematic diagram illustrating another exemplary gamma knife system according to some embodiments of the present disclosure.

The gamma knife system 550 shown in FIG. 5C is the same as or similar to the gamma knife system 500 shown in FIG. 5A. For example, the gamma knife system 550 shown in FIG. 5C may include a support, a gamma knife 510, and a magnetic resonance imaging (MRI) device 520. The support is not shown in FIG. 5C.

Unlike the gamma knife system 500, a location of the gamma knife 510 in the gamma knife system 550 is different from the location of the gamma knife 510 in the gamma knife system 500. That is, the gamma knife 510 in the gamma knife system 550 may be provided at a side of the MRI device 520, and the gamma knife 510 in the gamma knife system 500 may be provided at an upper end of the MRI device 520.

Some of the following embodiments may be understood with reference to FIG. 5C. However, the accompanying drawings are only a schematic illustration of some of these embodiments, and they do not constitute a limitation of the embodiments.

In some embodiments, as shown in FIG. 5C, the direction of the radiation beam of the radiotherapy device 510 may be perpendicular or approximately perpendicular to the direction of the magnet field of the magnet assembly 521, i.e., the direction of the radiation beam of the radiotherapy device 510 may be perpendicular or approximately perpendicular to the direction shown by H.

In some embodiments, the radiotherapy device 510 may rotate around the X-axis shown in FIG. 5C. In some embodiments, when the radiotherapy device 510 rotates around the X-axis shown in FIG. 5C, a treatment angle may be 360° in a traverse section of the subject (i.e., the plane in which the Y-axis and Z-axis are located). In some embodiments, when the radiotherapy device 510 rotates around the X-axis shown in FIG. 5C, the magnetic resonance imaging device 520 may rotate synchronously with the rotation of the radiotherapy device 510 to reduce the attenuation of the treatment radiation rays. In some embodiments, the radiotherapy device 510 may also rotate around the Y-axis as shown in FIG. 5C. In some embodiments, when the radiotherapy device 510 rotates around the Y-axis shown in FIG. 5C, the treatment angle may be in a plane perpendicular to the Y-axis shown in FIG. 5C (i.e., the plane where the X-axis and Z-axis are located). In this embodiment, the treatment angle may be less than 90°, which may reduce damage to other parts of the organs outside the subject (e.g., the mouth, nose, etc.), thus performing a more precise treatment on the subject.

In some embodiments, the radiotherapy device 510 may move in a direction in which the direction of the radiation beam is perpendicular to the direction of the magnet field and at an angle between the direction of the radiation beam and the Z-axis as shown in FIG. 5B within a range of 0°-45°. The rotation or movement of the radiotherapy device 510 in the gamma knife system 500 shown in FIG. 5C may be similar to the rotation or movement of the radiotherapy device 410 in the gamma knife system 400 shown in FIG. 4C, which may be described in detail in FIG. 4C and may not be repeated herein.

By setting the direction of the radiation beam of the radiotherapy device 510 to be perpendicular to the direction of the magnet field of the magnet assembly 521, the treatment radiation rays emitted by the radiotherapy device 510 may be unnecessary to pass through the gantry 522 or the magnet assembly 521. Thus, attenuation of the treatment radiation rays may not occur, and a guiding slot may not be required to be provided on the gantry 522, thereby reducing the manufacturing difficulty of the MRI device 520 and reducing the manufacturing process. Furthermore, the magnet field may be uniform, which allows for better online magnetic resonance imaging and online radiation treatment on the subject.

Since the treatment radiation rays emitted by the radiotherapy device 510 are unnecessary to pass through the gantry 522 or the magnet assembly 521, the direction of the radiation beam is perpendicular to the direction of the magnet field, and the magnet field has a greater influence on the radiation beam, measures are needed to be taken to reduce the influence of the magnet field on the radiation beam. In some embodiments, the gamma knife system 500 may also include a shielding structure (e.g., a magnetic shielding material, which is not shown in FIG. 5B) to reduce the effect of the magnet field on the radiation beam.

FIG. 6A is a schematic diagram illustrating an exemplary gamma knife system according to some embodiments of the present disclosure. FIG. 6B is a schematic diagram illustrating a section view of a gamma knife system as shown in FIG. 6A according to some embodiments of the present disclosure.

The gamma knife system 600 shown in FIG. 6A and the gamma knife system 400 shown in FIG. 4A and the gamma knife system 500 shown in FIG. 5A may be similar in structure and mainly different in the type of magnet.

Some of the following embodiments may be understood with reference to FIG. 6A. However, the accompanying drawings are only a schematic illustration of some of these embodiments and do not constitute a limitation of the embodiments.

In some embodiments, the gamma knife system 600 may include a radiotherapy device 610, a magnetic resonance imaging (MRI) device, and a support, as shown in FIG. 6A. In this embodiment, the support may be a rotating shell 630.

In some embodiments, the magnetic resonance imaging device may include a magnet and a gantry (not shown in FIG. 6A), with the magnet provided on the gantry. In some embodiments, the radiotherapy device 610 may rotate around at least one rotation axis. In some embodiments, the rotation axis may be perpendicular and/or parallel to a direction of a magnet field of the magnet. For example, the at least one rotation axis may be perpendicular and/or parallel to the direction of the magnet field in FIG. 6B (the direction shown by L in FIG. 6B).

In some embodiments, a three-dimensional coordinate system may be preset in the gamma knife system 600 shown in FIG. 6A. For example, a reference coordinate system may be constructed by taking a direction L (i.e. a direction parallel to a central axis along a center of an aperture of the cylindrical magnet) of the magnetic field of the cylindrical magnet as the X-axis (which may be perpendicular to a traverse section of a subject when lying in an accommodation space), taking a direction perpendicular to the direction L of the magnetic field of the cylindrical magnet and perpendicular to an extension direction of a table as the Y-axis (which may be perpendicular to a coronal plane of the subject when lying in the accommodation space), and taking a direction perpendicular to the direction L of the magnetic field of the cylindrical magnet and parallel to the extension direction of the table as the Z-axis (which may be perpendicular to a sagittal plane of the subject when lying in the accommodation space).

In some embodiments, the at least one rotation axis may include at least an X-axis perpendicular to the traverse section of the subject as shown in FIG. 6B, and the X-axis may be parallel to the direction L of the magnet field. In some embodiments, the rotation axis may further include a Z-axis as shown in FIG. 6B, and the Z-axis may be perpendicular to an axis of the rotating shell 630 and perpendicular to the direction L of the magnet field.

In some embodiments, the magnet assembly may be a cylindrical magnet 621, such as a cylindrical permanent magnet or a cylindrical electromagnet. In some embodiments, as shown in FIG. 6A, an interior of the cylindrical magnet 621 may have an accommodation space for accommodating the subject (e.g., the head of the patient). A magnet field in a horizontal direction (i.e., the extension direction of the table) may be formed in the accommodation space of the cylindrical magnet 621 (e.g., when the cylindrical magnet 621 is the cylindrical electromagnet), i.e., the direction shown by L in FIG. 6B may be the direction of the magnet field.

In some embodiments, a ray dose of the radiotherapy device 610 may be increased to ensure that the radiation beam from the radiotherapy device 610 may pass through the cylindrical magnet 621 to irradiate the subject.

In some embodiments, an angle between the direction of the radiation beam of the radiotherapy device 610 and the direction of the magnet field of the cylindrical magnet 621 (i.e., the direction shown by L) may be acute.

In some embodiments, the radiotherapy device 610 may rotate around the X-axis shown in FIG. 6B. In some embodiments, when the radiotherapy device 610 rotate around the X-axis shown in FIG. 6B, a treatment angle may be 360° in the traverse section of the subject (i.e., a plane in which the Y-axis and Z-axis are located). In some embodiments, when the magnetic resonance imaging device rotates synchronously with the rotation of the radiotherapy device 610 when the radiotherapy device 610 rotates around the X-axis as shown in FIG. 6B.

In some embodiments, the radiotherapy device 610 may rotate around the Z-axis shown in FIG. 6B. In some embodiments, when the radiotherapy device 610 rotates around the Z-axis shown in FIG. 6B, the treatment angle may be on the sagittal plane or the coronal plane of the subject (i.e., the plane in which the X-axis and Z-axis are located, or the plane in which the X-axis and Y-axis are located). In this embodiment, the treatment angle may be less than 90°, which may reduce damage to other parts of the organs outside the subject (e.g., the mouth, nose, etc.), and thus perform a more precise treatment on the subject.

In some embodiments, the radiotherapy device 610 may move at an angle between the direction of the radiation beam and the direction of the magnet field (i.e., the direction shown by L in FIG. 6B) within a range of 0°-45°. The rotation or movement of the radiotherapy device 610 in the gamma knife system 600 shown in FIG. 6A may be similar to the rotation or movement of the radiotherapy device 410 in the gamma knife system 400 shown in FIG. 4A. Detailed descriptions may be found in FIG. 4A and are not repeated here.

In some embodiments, the gamma knife system 600 may also include the rotating shell 630. The rotating shell 630 may be similar in structure to the rotating shell 430. More descriptions regarding the rotating shell 630 and the radiotherapy device 600 may be found in the related descriptions in FIG. 4A, which may not be repeated herein.

FIG. 6C is a schematic diagram illustrating an exemplary gamma knife system according to some embodiments of the present disclosure.

Some of the following embodiments may be understood with reference to FIG. 6C. However, the accompanying drawings are only a schematic illustration of some of the embodiments therein, and do not constitute a limitation of the embodiments.

The gamma knife system 650 may be same as or similar to the gamma knife system 600 as shown in FIG. 6A. For example, the gamma knife system 650 may include a radiotherapy device 610, a magnetic resonance imaging (MRI) device, and a support. Different from the gamma knife system 600, as shown in FIG. 6C, the MRI device mat include a cylindrical magnet 621. The cylindrical magnet 621 may include a first section 621-1 and a second section 621-2. The diameter of the first section 621-1 may be smaller than the diameter of the second section 621-2, and the first section 621-1 may be close to the radiotherapy device 610. By setting the diameter of the first section 621-1 smaller than the diameter of the second section 621-2, an internal magnetic density may be higher, and the magnetic resonance imaging effect may be better.

The first section 621-1 may be more adapted to a portion of a subject with a smaller size and the second section 621-2 may be more adapted to a portion of the subject with a larger size.

For example, as shown in FIG. 6C, the first section 621-1 may be configured to accommodate the head of the patient, and the second section 621-2 may be configured to accommodate the shoulder of the patient. In some embodiments, a ratio of the diameter of the first section 621-1 to the diameter of the second section 621-2 and/or a ratio of a length of the first section 621-1 to a length of the second section 621-2 may be set to different to adapt to different heads and body sizes of different patients.

In some embodiments, as shown in FIG. 6C, a total length of the first section 621-1 and the second section 621-2 may be greater than the length of each cylindrical magnet 621 in FIG. 6A, to improve the uniformity of a magnet field at the first section 621-1 to facilitate better magnetic resonance imaging of the head of the patient.

The gamma knife system 600 include the MRI device including the cylindrical magnet 621 which includes multiple segments with different diameters shown in FIG. 6C and the gamma knife system 600 shown in FIG. 6A may be similar in structure. Except for the cylindrical magnet 621, detailed descriptions regarding the structure may be found in the related descriptions in FIG. 6A and may not be repeated herein.

FIG. 6D is a schematic diagram illustrating an exemplary structure of a gamma knife system according to some embodiments of the present disclosure. FIG. 6E is a schematic diagram illustrating a traverse section of a gamma knife system according to some embodiments of the present disclosure.

The gamma knife system 600 shown in FIGS. 6D and 6E is an alternative implementation of FIG. 6C, which works similarly. The gamma knife system 600 shown in FIGS. 6D and 6E may be additionally provided with a third magnet 622, and the rest of the structure and relative positions are otherwise the same as that of the gamma knife system 600 of different diameters shown in FIG. 6C.

Some of the following embodiments may be understood with reference to FIGS. 6D and 6E, but the accompanying drawings are only an illustration of some of these embodiments and do not constitute a limitation of the embodiments.

Different from the gamma knife system 650, as shown in FIGS. 6D and 6E, the MRI device may include a third magnet 622 and one or more magnets each of which is in a tubular structure (also referred as cylindrical magnet 621), and the third magnet 622 and the cylindrical magnet 621 may be provided on two sides of a radiotherapy device 610 and the rotating shell 630, and the support along a direction of the magnet field, wherein the cylindrical magnet 621 is used to form the accommodation space, and the shape of the third magnet 622 is not limiting. In some embodiments, as shown in FIGS. 6D and 6E, the cylindrical magnet 621 includes the first section 621-1 and the second section 621-2 may be provided on a side facing a direction of a radiation beam of the radiotherapy device 610, and the third magnet 622 may be provided on a side away from the direction of the radiation beam of the radiotherapy device 610.

The gamma knife system 600 shown in FIGS. 6D and 6E and the gamma knife system 600 shown in FIG. 6C of different diameters may be similar in structure. Except for the cylindrical magnet 621, detailed descriptions regarding the structure may be found in related descriptions in FIG. 6A and FIG. 6C and may not be repeated here.

By providing the third magnet 622 on a side near the first section 621-1 and away from the second section 621-2, a higher magnetic density and a better uniformity of the magnet field inside the first section 621-1 may be achieved. In such a case, imaging of the head of the patient may be better, thereby facilitating online treatment on the head of the patient by the radiotherapy device 610.

It should be noted the above descriptions are for illustration purpose and non-limiting. In some embodiments, a magnet field in a perpendicular direction (i.e., a direction perpendicular to the extension direction of the table) may be formed in the accommodation space of the cylindrical magnet 621 (e.g., when the cylindrical magnet 621 is the cylindrical permanent magnet), i.e., the direction shown by Y in FIG. 6B may be the direction of the magnet field.

Some embodiments of the present disclosure may produce, but are not limited to, the following beneficial effects. (1) By combining the MRI device and the radiotherapy device into an integrated device, the radiotherapy device may rotate around a rotation axis perpendicular to and/or parallel to the direction of the magnet field of the magnetic resonance imaging device. That is to say, the radiotherapy device may rotate synchronously with the rotation of the MRI device, and the radiotherapy device may rotate relative to the rotation of the magnetic resonance imaging device, to make the MRI device and the radiotherapy device interlinked and controlled. The MRI device has the advantage of high imaging contrast, which enables online localization of the subject (e.g., a lesion), and the radiotherapy device may perform online treatment on the subject based on a magnetic resonance image. (2) By setting the radiotherapy device to rotate around an axis perpendicular to the traverse section of the subject, treatment at an angle of 360° on the traverse section of the subject is realized. In combination with the setting of the angle between the direction of the radiation beam of the radiotherapy device and the direction of the magnet field of the magnet to be acute, online three-dimensional angular treatment (i.e., treatment at an angle of 360° on the traverse section and treatment at an angle less than 90° on the sagittal plane or the coronal plane) is realized, making the treatment on the subject more precise. Further, the use of different ray doses at different angles between the direction of the radiation beam and the magnet field direction may simultaneously reduce damage to other parts of the body (e.g., the mouth, the nose, etc.). (3) By choosing a plurality of structural forms such as a permanent magnet, a conventional or superconducting magnet, an upper and lower open magnet, a horizontal cylindrical magnet, etc., to manufacture the gamma knife system guided by magnetic resonance, the system may be adapted to different application scenarios. It should be noted that the beneficial effects that may be produced in different embodiments may vary, and the beneficial effects that may be produced in different embodiments may be any one or a combination of any of the foregoing, or any other beneficial effect that may be obtained.

In addition, some features, structures, or characteristics of one or more embodiments in the present disclosure may be properly combined.

In some embodiments, the numbers expressing quantities or properties used to describe and claim certain embodiments of the present disclosure are to be understood as being modified in some instances by the term “about,” “approximate,” or “substantially.” For example, “about,” “approximate” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

By way of example, should there be any inconsistency or conflict between the description, definition, and/or the use of a term associated with any of the incorporated material and that associated with the present document, the description, definition, and/or the use of the term in the present document shall prevail.

What is claimed is:

1. A treatment system, comprising:
a radiotherapy device configured to emit a radiation beam;
a magnetic resonance imaging (MRI) device including a magnet assembly for providing a magnet field in an accommodation space; and
a support configured to be connected with the radiotherapy device, wherein the radiotherapy device is configured to rotate around at least one rotation axis.

2. The treatment system of claim 1, wherein an angle between a direction of the magnet field in the accommodation space and a direction of the radiation beam is equal to or less than 90 degrees.

3. The treatment system of claim 1, wherein the at least one rotation axis includes at least one of a first axis parallel with an extension direction of a table of the MRI device in the accommodation space or a second axis perpendicular to the extension direction of the table of the MRI device in the accommodation space.

4. The treatment system of claim 3, wherein a rotation angle of the radiotherapy device rotating around the first axis is 360 degrees.

5. The treatment system of claim 3, wherein the radiotherapy device is configured to rotate around the first axis with a rotation of the support.

6. The treatment system of claim 3, wherein the radiotherapy device is configured to rotate around the first axis with a rotation of the MRI device.

7. The treatment system of claim 1, wherein the radiotherapy device is configured to rotate around one of the at least one rotation axis independently of the support.

8. The treatment system of claim 3, wherein a rotation angle of the radiotherapy device rotating around the second axis is less than 180 degrees.

9. The treatment system of claim 3, wherein the second axis is perpendicular or parallel to a surface of the table where a subject to be treated, and the radiotherapy device is arranged on the support such that a direction of the radiation beam is perpendicular or substantially perpendicular to a plane defined by the second axis and the first axis.

10. The treatment system of claim 3, wherein the at least one axis includes a third axis, an angle between the third axis and the second axis is less than 90 degrees.

11. The treatment system of claim 1, wherein the support includes a rotating shell, wherein the rotating shell in a semicircular shape is configured to provide the support to the radiotherapy device.

12. The treatment system of claim 1, wherein the magnet assembly includes a first magnet and a second magnet separately provided at two sides of the accommodation space along a direction parallel to a direction of the magnet field.

13. The treatment system of claim 12, wherein a direction of the radiation beam is parallel to or substantially parallel to the direction of the magnet field.

14. The treatment system of claim 12, wherein a direction of the radiation beam is perpendicular to or substantially perpendicular to the direction of the magnet field.

15. The treatment system of claim 12, wherein the at least one rotation axis includes a first axis perpendicular to a direction of the magnet field in the accommodation space and a second axis perpendicular to or parallel with the direction of the magnet field in the accommodation space.

16. The treatment system of claim 12, wherein a guiding slot is provided on a gantry of the MRI device and configured to pass the radiation beam, and/or a recess is provided on each of the first magnet and the second magnet which are configured to pass the radiation beam.

17. The treatment system of claim 1, wherein the magnet assembly includes one or more magnets each of which is in a tubular structure and surrounds the accommodation space.

18. The treatment system of claim 17, wherein the at least one rotation axis includes a first axis parallel with a direction of the magnet field in the accommodation space and a second axis perpendicular to the direction of the magnet field in the accommodation space.

19. The treatment system of claim 17, wherein the magnet assembly includes a first section and a second section, a size of the first section is smaller than a size of the second section.

20. The treatment system of claim 1, wherein the magnet assembly includes one or more magnets each of which is in a tubular structure to form the accommodation space and a third magnet, the one or more magnets and the third magnet are located on two sides of the radiotherapy device and the support along a direction of the magnet field.
